(19) United States
(12) Patent Application Publication
(10) Pub. No.: US 2019/0351003 A1
(43) Pub. Date: Nov. 21, 2019

(54) METHODS FOR INHIBITING NONALCOHOLIC STEATOHEPATITIS, NONALCOHOLIC FATTY LIVER DISEASE, AND/OR DE NOVO LIPOGENESIS

(71) Applicant: The Regents of the University of California, Oakland, CA (US)

(72) Inventors: Michael Karin, La Jolla, CA (US); Juyoun Kim, San Diego, CA (US)

(21) Appl. No.: 16/483,583

(22) PCT Filed: Feb. 7, 2018
(86) PCT No.: PCT/US2018/017177

§ 371 Date: Aug. 5, 2019

Related U.S. Application Data
(60) Provisional application No. 62/455,993, filed on Feb. 7, 2017.
(60) Provisional application No. 62/537,703, filed on Jul. 27, 2017.

(51) Int. Cl. A61K 38/00; C07K 7/06; C12N 15/86; C12N 9/50; A61P 1/16

(52) U.S. Cl. CPC A61K 38/005; C07K 7/06; C12Y 304/22055; C12N 9/50; A61P 1/16; C12N 15/86

(57) ABSTRACT

Provided herein are methods of treating nonalcoholic steatohepatitis (NASH), nonalcoholic fatty liver disease (NAFLD), and/or elevated de novo lipogenesis (DNL) by inhibiting caspase-2 activity or expression. Also disclosed are methods of screening for agents useful in such methods.

### CROSS REFERENCE TO RELATED APPLICATION(S)

This application claims the benefit of priority under 35 U.S.C. § 119(e) of U.S. Ser. No. 62/455,993, filed Feb. 7, 2017, and of U.S. Ser. No. 62/537,703, filed Jul. 27, 2017, the entire content of each of which is incorporated herein by reference.

### GRANT INFORMATION

This invention was made with government support under Grant Nos. CA155120, ES010337, and CA118165 awarded by the National Institutes of Health. The United States government has certain rights in the invention.

### BACKGROUND OF THE INVENTION

### Field of the Invention

The invention relates generally to liver diseases and more specifically to methods of treating nonalcoholic fatty liver disease, liver steatohepatitis, de novo lipogenesis, and/or hepatocellular carcinoma caused by nonalcoholic steatohepatitis.

### Background Information

The obesity epidemic currently afflicting the US and other developed countries has resulted in a marked increase in the incidence of the metabolic syndrome and its associated pathologies, including nonalcoholic fatty liver disease (NAFLD), estimated to affect 30% of Americans (Cohen et al., 2011; Farrell et al., 2012). Although NAFLD is characterized by hepatocyte lipid droplet buildup, only in 15-20% of patients is it accompanied by liver damage, inflammation, and fibrosis, the hallmarks of nonalcoholic steatohepatitis (NASH). Development of NASH depends on secondary factors, including endoplasmic reticulum (ER) stress and mitochondrial dysfunction (Tilg and Moschen, 2010). In the context of simple, non-symptomatic liver steatosis, ER stress or mitochondrial dysfunction trigger nonalcoholic steatohepatitis (NASH), a serious disease that can progress to liver cirrhosis, resulting in loss of liver function, and hepatocellular carcinoma (HCC), one of the most deadly cancers.

It is estimated that at least 10-15% of NAFLD patients eventually progress to NASH (Cohen et al., 2011; Farrell et al., 2012), which is predicted to quickly become the leading cause of liver transplantation and HCC in the U.S. Although the subject of intense efforts in academia and pharma alike, no effective interventions that treat or prevent NASH exist, other than caloric restriction, exercise, or bariatric surgery, which are successful only when applied early. A need therefore exists for treatments that prevent or ameliorate NASH and attenuate its progression to HCC.

### SUMMARY OF THE INVENTION

The present invention relates to the identification of a biochemical pathway responsible for activation of SREBP1/2 in mice suffering from NASH and demonstrates that the same pathway is active in human NASH patients. The data presented herein demonstrates that inhibition of a critical component of this pathway reverses all NASH-related symptoms in mice and prevents fat accumulation in liver and enhances energy expenditure.

Accordingly, the invention provides a method of treating or preventing nonalcoholic steatohepatitis (NASH), nonalcoholic fatty liver disease (NAFLD), and/or de novo lipogenesis (DNL) in a subject in need thereof. The method includes administering to the subject an effective amount of an inhibitor of caspase-2 activity or expression. Thus, in various embodiments, the invention provides use of an inhibitor of caspase activity or expression in the treatment of nonalcoholic steatohepatitis (NASH), nonalcoholic fatty liver disease (NAFLD), and/or de novo lipogenesis (DNL) in a subject. In various embodiments, the inhibitor of caspase-2 activity or expression is selected from the group consisting of IDN-6556, VDVAD, DARPin, Ac-VDVAD-CHO (C₂₃H₃₇N₅O₁₀), z-VDVAD-FMK (C₃₂H₄₆FN₅O₁₁), and Z-FA-FMK (C₂₁H₂₃FN₂O₄). In various embodiments, the inhibitor of inhibitor of caspase-2 activity or expression is an inhibitory nucleic acid that inhibits the expression of casp2. For example, the inhibitory nucleic acid can be siRNA, shRNA, guide RNA (gRNA), oligonucleotides, antisense RNA or ribozymes that inhibit casp2 synthesis. As appropriate, the inhibitory nucleic acid can be delivered in a viral vector, for example, lentiviral vector, a herpesvirus vector or an adenoviral vector.

In another aspect, the invention provides a method of identifying an agent useful for treating and/or preventing NASH, NAFLD, DNL and/or hepatocellular carcinoma caused by nonalcoholic steatohepatitis. The method includes contacting a sample of cells with at least one test agent, wherein a decrease in caspase-2 activity or expression, or expression of a caspase-2 stimulated reporter gene, in the presence of the test agent as compared to caspase-2 activity or expression, or expression of a caspase-2 stimulated reporter gene, in the absence of the test agent identifies the agent as useful for treating or inhibiting hepatocellular carcinoma, NASH, NAFLD, and/or DNL. In various embodiments, the test agent is an inhibitor of caspase-2 activity or expression. In various embodiments, the method may be performed in a high throughput format, such as contacting samples of cells of a plurality of samples with at least one test agent. In various embodiments, the plurality of samples may be obtained from a single subject or from different subjects.

In another aspect, the invention provides a method identifying NASH-induced hepatocellular carcinoma amenable to treatment with an inhibitor of caspase-2. The method includes detecting elevated caspase-2 activity or expression in a sample of cells as compared to caspase-2 activity or expression in corresponding normal cells, thereby identifying hepatocellular carcinoma amenable to treatment with an inhibitor of caspase-2. In various embodiments, the cells are from a biopsy sample obtained from a subject. In various embodiments, the cells are from a tissue or bodily fluid obtained from a subject.

### BRIEF DESCRIPTION OF THE DRAWINGS

FIGS. 1A-1D are pictorial and graphical diagrams showing that Caspase 2 is required for NASH development. FIG. 1A shows liver gross morphology (bar: 1 cm) and immunohistochemical (IHC) analysis of FFPE sections from livers of indicated mouse strains. H&E staining was used to reveal tissue histology, macrovesicular fats and hepatocyte ballooning. Immunostainings for p62 and F4/80 were used to examine Mallory Denk Body (MBD) formation and macrophage infiltration, respectively. Oil Red O was used to visualize lipid droplets. Scale bars: 100 μm. Staining intensity per high magnification field (HMF) was determined by Image J analysis of 20 fields for each section (MUP: n=8; Casp2^{-/-}/MUP: n=6). Bar graphs represent averages±SEM. FIG. 1B shows that liver Casp2 mRNA content (arbitrary units, A.U.) was determined by qRT-PCR analysis (MUP: n=7; Tnfr1^{-/-}/MUP: n=8). FIG. 1C shows serum and liver triglycerides (TG) and cholesterol (Chol) in indicated mouse strains (WT/MUP: n=10; Casp2^{-/-}/MUP: n=7). FIG. 1D shows Casp2 expression in liver biopsies from NAFLD (n=4) and NASH (n=4) patients. Staining intensity was quantified by Image J analysis. Bar: 100 μm. Bar graphs represent averages±SEM. *P<0.05 **P<0.005 ***P<0.001.

FIGS. 2A-2D are pictorial and graphical diagrams showing that Caspase 2 controls adipose tissue expansion in response to hypernutrition. FIG. 2A shows body weight and bi-weekly food intake of indicated mouse strains kept on HFD for 12 weeks (MUP: n=12, Casp2^{-/-}/MUP: n=10). Averages±SEM. FIG. 2B shows gross morphology and weights of epidydimal fat from HFD-fed mice (MUP: n=12; Casp2^{-/-}/MUP: n=10). FIG. 2C shows that FFPE sections of epidydimal fat were H&E stained (magnification bar: 100 pin). Adipocyte area/HMF, adipocyte number/HMF and adipocyte size distributions were determined by Image J analysis of 5 HMF per section (MUP: n=7, Casp2^{-/-}/MUP: n=9). FIG. 2D shows cytokine and inflammation marker mRNAs were quantified by qRT-PCR analysis of epidydimal fat RNA. Bar graphs averages±SEM. *P<0.05 **P<0.005 ***P<0.001.

FIGS. 3A-3D are pictorial and graphical diagrams showing that Caspase 2 ablation increases energy expenditure in MUP-uPA mice. FIG. 3A shows VO₂, VCO₂, and RER measurements in indicated mice strains that were placed on HFD for 12 weeks. The different parameters were recorded for 2 days composed of 2 dark and light cycles (n=6/group). Averages±SEM. FIGS. 3B and 3D show p-AMPK and AMPK were examined by IB analysis of liver, skeletal muscle and adipocyte extracts from the indicated mouse strains. FIG. 3C shows UCP1 mRNA in inguinal fat (iWAT) and brown adipose tissue (BAT) of indicated mouse strains was determined by qRT-PCR analysis (MUP: n=12, Casp2^{-/-}/MUP: n=10). Averages±SEM. *P<0.05 **P<0.005 ***P<0.001.

FIGS. 4A-4K are pictorial and graphical diagrams showing that Caspase 2 controls SREBP activation and de novo lipogenesis. FIG. 4A shows the results from IB analysis of SREBP1/2 and Casp2 in whole cell lysates (WCL) and nuclear extracts (NE) from livers of 5-week-old MUP-uPA and Casp2^{-/-}/MUP-uPA mice. P-precursor, C-cleaved, N-nuclear. FIG. 4B shows the results of IB analysis of SREBP1/2 in liver WCL and NE of from HFD-fed 20-week-old mice of indicated genotypes. FIG. 4C shows the results of qRT-PCR analysis of mRNAs encoding lipogenic proteins in total liver RNA from HFD-fed mice of indicated genotypes. FIG. 4D shows the results of IB analysis of indicated proteins in differentiated adipocyte WCL from indicated mouse strains. FIG. 4E shows that de novo lipogenesis was measured by ¹⁴C-incorporation into newly synthesized lipids in adipocytes incubated with BSA or insulin. Preadipocytes were differentiated for 6 days (n=3). Averages±SEM). *P<0.05 **P<0.005 ***P<0.001. FIG. 4F shows the results of IB analysis of SREBP1/2 in liver nuclear extracts (NE) of liver obtained from WT BL6 and MUP-uPA mice that were HFD-fed for 12 weeks. FIG. 4G shows the results of qRT-PCR analysis of mRNAs encoding lipogenic proteins in total liver RNA from HFD-fed indicated mice. Mice were HFD-fed for 12 weeks (BL6: n=5; MUP: n=8; Casp2^{-/-}/MUP-uPA: n=8). Mean±SEM. FIG. 4H shows that StARD4 was detected by IB analysis in liver WCL of 5-week-old indicated mice. FIG. 4I shows the results of IB analysis of SCAP and INSIG1 in liver WCL from the indicated mouse strains that were HFD-fed for 12 weeks. FIG. 4J shows that primary hepatocytes of the indicated genotypes were incubated with either PBS or TNF for 8 hr. Membranes and NE were prepared and IB-analyzed with the indicated antibodies. FIG. 4K shows that primary Casp2^{-/-} hepatocytes were transduced with either control or Casp2-encoding lentiviruses. After 36 hr, the hepatocytes were incubated with either PBS or TNF for 3 hr, membrane fractions and NE were prepared and subjected to IB analysis.

FIGS. 5A-5J are pictorial and graphical diagrams showing that Caspase 2 cleaves SIP to initiate SREBP processing. FIG. 5A shows indicated expression vectors were transfected into WT HEK293 cells. After 48 hr, cells were incubated with 25 μg/ml N-acetyl-leucine-leucine-norleucinal (ALLN) for 3 hr. WCL were IB analyzed with antibodies to SREBP1 and 2. FIG. 5B shows 293^{ΔSCAP} cells were transiently transfected with expression vectors for the indicated proteins and incubated in 1% LPDS for 16 hr. The cells were further treated with 25 μg/ml ALLN for 3 hr and membranes and NE were prepared. SREBP2 processing and protein loading were examined by IB analysis. FIG. 5C shows indicated expression vectors were transfected into WT HEK293 cells. After 5 hr, the cells were incubated with 1% lipoprotein-deficient serum (LPDS) for 12 hr, followed by ethanol or 50 μM mevalonate plus 5 μg/ml cholesterol for 12 hr. 3 hr before harvest, cells were treated with 25 μg/ml ALLN. WCL, membranes, and NE were prepared and immunoblotted for indicated proteins. Red stars: the N-terminal Myc epitope containing SIP polypeptides. FIG. 5D shows 293^{ΔSCAP} cells were transiently transfected with expression vectors for the indicated proteins and incubated in 1% LPDS for 16 hr. WCL were prepared and analyzed by IB for presence of cleaved Casp3 (Cl.Casp3). WCL prepared from WT mouse organoids treated with TNF (40 ng/ml) and cycloheximide (10 μg/ml) for 2 hr were used as positive controls. FIG. 5E shows 293^{ΔSCAP} cells were transfected with the indicated expression vectors. After 24 hr, cells were incubated with DMSO or tunicamycin (1 μg/ml) for 6 hr and 25 μg/ml ALLN for 3 hr before harvest. Membranes and NE were prepared and IB-analyzed with the indicated antibodies. S.E: short exposure, L.E: long exposure. FIG. 5F shows the indicated proteins were transiently expressed in WT HEK293 cells. After 48 hr, WCL and culture supernatants (CS) were immunoblotted with Myc and SIP antibodies (2 lanes per condition). FIG. 5G shows the indicated proteins were transiently expressed in SCAP-ablated HEK293 cells. 5 hr after transfection, the cells were incubated with 1% LPDS for 16 hr followed by 3 hr treatment with ALLN. WCL, membranes, and NE were prepared and Casp2 and SP were immunoblotted with HA and Myc antibodies. CS were IB analyzed for SIP. Arrow: cleaved Casp2, red stars: N-terminal SP fragments that retained the Myc epitope. FIG. 5H shows the ER and Golgi compartments were isolated by differential centrifugation from livers of LFD-fed 7-week-old MUP-uPA and Casp2^{-/-}/MUP-uPA mice. Proteins obtained from each compartment were de-glycosylated with PNGase F and immunoblotted as indicated. Star: nonspecific band present in the Golgi fraction of Casp2-null liver, F.L: full-length, C2-Cl.: cleaved by Casp2, A-Cl.: autocleaved. FIG. 5I shows the results from IB analysis of SIP in serum of HFD-fed (12 weeks) mice of the indicated genotypes. The splice mark indicates removal of an irrelevant lane. FIG. 5J shows sera from normal individuals, NAFLD and NASH patients (n=3 per group) with different liver fibrosis scores were immunoblotted with SP antibody.

FIGS. 6A-6G are pictorial and graphical diagrams showing that Casp2 inhibition ameliorates NASH. FIG. 6A shows an exemplary experimental scheme. After 6 weeks of HFD feeding, MUP-uPA mice were treated with Ac-VDVAD (10 μg/g) for 6 weeks while kept on HFD. FIG. 6B shows that pharmacological inhibition of Casp2 ameliorates established NASH. FFPE sections from livers of inhibitor or vehicle treated mice were evaluated for macrovesicular fat, ballooning hepatocytes, MDB and p62 aggregates, macrophages, collagen fibers and lipid droplets. Magnification bars: 100 μm. Images were quantitated as above and results are shown on the right as averages±SEM (Vehicle: n=6; Ac-VDVAD: n=5). FIG. 6C shows the results from qRT-PCR analyses of inflammatory and fibrogenic mRNAs (Vehicle: n=6; Ac-VDVAD: n=7). Mean±SEM. FIG. 6D shows TG and cholesterol concentrations in serum and liver of treated mice (Vehicle: n=6; Ac-VDVAD: n=7). FIG. 6E shows that adipocytes were visualized by H&E staining of FFPE epidydimal fat sections from inhibitor- or vehicle-injected mice. Adipocyte size and density were determined as above. ATM (adipose tissue macrophages) were visualized by F4/80 staining and quantified as above. FIG. 6F shows that SREBP1/2 activation was IB analyzed in WCL and NE from livers of untreated and treated mice. FIG. 6G shows that AMPK phosphorylation was analyzed by IB analysis of liver and muscle extracts.

FIGS. 7A-7D are pictorial and graphical diagrams. FIG. 7A shows liver gross morphology (bar=4 mm) and IHC analysis of FFPE liver sections. The sections were stained with H&E, Oil Red O and p62 and F4/80 antibodies. FIG. 7B shows serum fatty acid (FA) composition in 6 month-old mice of the indicated genotypes that were kept on HFD for 20 weeks. Results are averages±SEM (MUP:n=5; Tnfr1^{-/-}/MUP:n=8; Casp2^{-/-}/MUP:n=6) *P<0.05, **P<0.005, ***P<0.001. FIG. 7C shows liver free cholesterol (Chol) in the indicated mouse strains kept on HFD for 12 weeks (BL6: n=5, MUP: n=10; Casp2^{-/-}/MUP: n=6). Mean±SEM. *p<0.05, **p<0.005, ***p<0.001. FIG. 7D shows expression of CHOP protein and uPA mRNA in livers of 5-week-old MUP-uPA and Casp2^{-/-}/MUP-uPA mice (n=3 per group).

FIGS. 8A-8D are pictorial and graphical diagrams. FIG. 8A shows body weight (BW) of mice of the indicated genotypes that were kept on HFD for 12 weeks. Each dot represents mean BW (WT BL6:n=3; MUP:n=12; Casp2^{-/-}/BL6:n=6; Casp2^{-/-}/MUP:n=6). FIG. 8B shows weights of epidydimal fat from HFD-fed mice were shown (WT BL6: n=6; Casp2^{-/-}/BL6:n=10). FFPE sections of epidydimal fat from WT BL6 and Casp2 mice were stained with H&E. Magnification bars: 100 μm. FIGS. 8C and 8D show the results from IB analysis of C/EBPβ and PPARγ expression and qRT-PCR analysis of c/Ebpb, Pparg, Srebf1, Srebf2, Adrp30, and Fabp4 mRNAs in adipocytes after two (FIG. 8C) or four (FIG. 8D) days of differentiation. Bar graphs: averages±SEM (n=3 per genotype).

FIG. 9 is a series of pictorial and graphical diagrams. The indicated mice were fed with HFD for 12 weeks and subjected to 4° C. cold challenge for 5 hrs and UCP1 protein and mRNA expression in BAT were examined by IB (left) and qRT-PCR (right) analyses. (MUP: n=8; Casp2^{-/-}/MUP: n=5).

FIGS. 10A-10H are pictorial and graphical diagrams. FIG. 10A shows a schematic representation of SIP primary structure, locations of actual and putative proteolytic cleavage sites for SIP itself and Casp2 and the different fragments they should generate. Bar: subtilisin homology domain; black bar: transmembrane domain. Dots: Asp, His, and Ser in the catalytic triad. Light bar: signal peptide. FIG. 10B shows Myc-S1P or empty vector was transiently transfected into 293^{ΔSCAP} cells and WCL were prepared and used for IB analysis. Intracellular SIP fragments were detected using Myc antibody. FIG. 10C shows a schematic representation of cDNAs encoding WT Casp2, catalytically inactive Casp2 (Casp2^{C320G}), SIP that is tagged with Myc epitope at either amino acid 23 to 24 (Myc-S1P) or at the carboxy terminus (S1P-Myc), SP mutant forms, SREBP1 and SREBP2. Intracellular SW was detected by an SW antibody that recognizes the first 50 amino acids and membrane and secreted SP polypeptides were detected by an antibody that recognizes amino acid 200 to amino acid 300. FIG. 10D shows the indicated proteins were transiently expressed in 293^{ΔSCAP} cells. 5 hr after transfection, the cells were incubated with 1% LPDS for 16 hr. WCL were prepared, gel separated and Casp2 and SP were detected by IB analysis with HA and Myc antibodies. CS were IB analyzed for SIP. The stars indicate processed SIP fragments, 100 kDa and 68 kDa in CS and ˜30 kDa in WCL. The arrow indicates cleaved Casp2. FIG. 10E shows the indicated proteins were transiently expressed in 293^{ΔSCAP} cells. 5 hr after transfection, the cells were incubated in 1% LPDS for 16 hr followed by 3 hr treatment with ALLN. Membranes and NE were prepared and SREBP1, INSIG1, PDI, and lamin B were detected by IB analysis. FIG. 10F shows indicated expression vectors were transfected into 293^{ΔSCAP} cells. After 5 hr, the cells were incubated in 1% LPDS for 12 hr, followed by addition of ethanol or 50 μM mevalonate plus 5 μg/ml cholesterol in ethanol for 12 hr. 3 hr before harvest, cells were treated with 25 μg/ml ALLN. Membranes and NE were prepared and the indicated proteins were IB analyzed. FIG. 10G shows the results of IB analysis of S1P in WCL and membrane fractions prepared from livers of 5-week-old mice of indicated genotypes. Cleaved SP in WCL was detected by anti-S1P antibody recognizing the first 50 N-terminal amino acid residues (#sc-271916, Santa Cruz Technologies), whereas membrane-associated full-length SIP was detected by anti-S1P that recognizes the catalytic pocket (#Ab140592, Abcam). FIG. 10H shows the indicated proteins were transiently expressed in WT HEK293 and 293^{ΔS2P} cells and SREBP2 processing was analyzed as in FIG. 5A.

FIGS. 11A and 11B are pictorial and graphical diagrams. FIG. 11A shows the results of IB analysis of SP in membrane fractions from livers of HFD-fed (12 weeks) mice of indicated genotypes. FIG. 11B shows that HEK 293T cells were transfected with indicated proteins. SREBP2 cleavage in response to co-expression of Casp2 with SP was examined by IB analysis in WCL. Processed/secreted SP was detected in media.

FIGS. 12A-12I are pictorial and graphical diagrams showing elevated Casp2 expression in ER-stressed mice and human NASH. FIG. 12A the results from immunoblot (IB) analysis of whole cell lysates (WCL) from 5-week-old WT and MUP-uPA livers (left) and WCL of livers from 3-month-old WT mice that were i.p. injected with dextrose or tunicamycin (1.25 mg/kg). Results are mean±SEM (dextrose: n=4; tunicamycin: n=5). P: precursor, C: cleaved. FIG. 12B shows that Casp2 mRNA in livers of above 5-week-old mice were quantitated by qRT-PCR. Results are mean±SEM (WT: n=5; MUP: n=9). FIG. 12C shows Casp2 mRNA in livers of 20-week-old MUP-uPA and Tnfr1^{-/-}/MUP-uPA mice kept on HFD for 12 weeks. Results are mean±SEM (MUP (LFD: n=4; HFD: n=8); Tnfr1^{-/-}/MUP (LFD: n=4; HFD: n=9)]. FIG. 12D shows liver Casp2 mRNA induction by tunicamycin in WT mice; results are mean±SEM (dextrose: n=4; tunicamycin: n=5). FIG. 12E shows CASP2 expression in liver biopsies from NAFLD (n=4) and NASH (n=4) patients. Staining intensity was quantified by ImageJ. FIG. 12F shows CASP2 and ER stress marker transcripts (HSPA5, DDIT3 and ATF6) in liver biopsies from NAFLD (n=10) or NASH (n=9) patients quantified by qRT-PCR. *p<0.05, **p<0.005, ***p<0.001. A.U., arbitrary units. FIG. 12E shows that Casp2, Casp3, and Casp8 expression was analyzed by IB of liver lysates obtained from the indicated mice at 5 weeks of age (first 2 lanes) or at 16 weeks after 8 weeks on LFD or HFD. Casp4 and Casp12 were examined in liver lysates of 5-week-old mice. FIG. 12F shows FFPE liver sections of 5-week-old mice on NC and 5-month-old mice that were kept on HFD for 12 weeks were stained with a Casp2 antibody (BL6: n=3; MUP: n=3). Staining intensity was quantitated using ImageJ as described in FIG. 1. FIG. 12G shows Casp2 and Tnf mRNA amounts in above livers were determined by qRT-PCR analysis of liver RNA from indicated mice (BL6: n=3; MUP: n=3). *p<0.05, **p<0.005, ***p<0.001.

FIGS. 13A and 13B are pictorial diagrams. FIG. 13A shows liver WCL prepared from either vehicle- or Ac-VDVAD-treated MUP-uPA mice were IB analyzed for the indicated proteins. Body weight and blood insulin of the same mice were measured and are shown on the right (vehicle: n=6; Ac-VDVAD: n=7). Mean±SEM. FIG. 13B shows WCL and FFPE BAT sections were prepared from vehicle- or Ac-VDVAD-treated MUP-uPA mice and UCP1 expression was analyzed by IHC and IB.

FIGS. 14A and 14B are pictorial diagrams showings results of AMPK activation in skeletal muscle and liver of VDVAD-injected MUP-uPA mice.

### DETAILED DESCRIPTION OF THE INVENTION

A common malignancy linked to chronic tissue damage, stress, and environmental carcinogen exposure is HCC, most of which arises as the end stage of chronic liver disease and persistent inflammation, with hepatitis B or C virus (HBV, HCV) infections being the current leading causes (El-Serag, 2011). However, obesity and alcohol consumption, which cause hepatic steatosis (non-alcoholic steatohepatitis (NASH) or alcoholic steatohepatitis (ASH), respectively) and fibrosis, are rapidly growing in their importance as HCC risk factors.

Endoplasmic reticulum (ER) stress has been implicated in the pathogenesis of viral hepatitis, insulin resistance, alcoholic hepatosteatosis (ASH) and non-alcoholic steatohepatitis (NASH), disorders that increase risk of hepatocellular carcinoma (HCC). The major process that contributes to fat accumulation in liver and visceral adipose tissue in non-alcoholic fatty liver disease (NAFLD), including its most severe manifestation, NASH, is de novo lipogenesis (DNL) and accumulation of free cholesterol. DNL and cholesterol accumulation depend on activation of transcription factors SREBP1 and SREBP2. The present disclosure identifies a novel biochemical pathway responsible for activation of SREBP1/2 in mice suffering from NASH and demonstrates that the same pathway is active in human NASH patients. The data presented herein demonstrates that inhibition of a critical component of this pathway prevents NASH development and reverses all NASH-related symptoms in mice by preventing de novo lipogenesis in liver and enhancing energy expenditure, thereby abrogating liver fat accumulation.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

As used herein, a “non-human mammal” may be any as long as it is other than human, and includes a transgenic animal and animals for which a production method of ES cells and/or iPS cells has been established. For example, rodents such as mouse, rat, hamster, guinea pig, rabbit, swine, bovine, goat, horse, sheep, dog, cat, and monkey are envisioned as non-human mammals.

The terms “administration” or “administering” are defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration or administration via intranasal delivery.

As used herein, an “effective amount” is an amount of a substance or molecule sufficient to effect beneficial or desired clinical results including alleviation or reduction in any one or more of the symptoms associated with cancer, NASH or hepatic steatosis. For purposes of this invention, an effective amount of a compound or molecule of the invention is an amount sufficient to reduce the signs and symptoms associated with cancer, such as hepatocellular carcinoma, NASH, hepatic steatosis. In some embodiments, the “effective amount” may be administered before, during, and/or after any treatment regimens for cancer or NASH.

As used herein, “treatment” is an approach for obtaining beneficial or desired clinical results. For purposes of this invention, beneficial or desired clinical results include, but are not limited to, treatment of cancer, such as hepatocellular carcinoma, NASH, or hepatic steatosis.

As used herein, the term “genetic modification” is used to refer to any manipulation of an organism's genetic material in a way that does not occur under natural conditions. Methods of performing such manipulations are known to those of ordinary skill in the art and include, but are not limited to, techniques that make use of vectors for transforming cells with a nucleic acid sequence of interest. Included in the definition are various forms of gene editing in which DNA is inserted, deleted or replaced in the genome of a living organism using engineered nucleases, or “molecular scissors.” These nucleases create site-specific double-strand breaks (DSBs) at desired locations in the genome. The induced double-strand breaks are repaired through nonhomologous end-joining (NHEJ) or homologous recombination (HR), resulting in targeted mutations (i.e., edits). There are several families of engineered nucleases used in gene editing, for example, but not limited to, meganucleases, zinc finger nucleases (ZFNs), transcription activator-like effector-based nucleases (TALEN), and the CRISPR-Cas system.

A “test agent” or “candidate agent” refers to an agent that is to be screened in one or more of the assays described herein. The agent can be virtually any chemical compound. It can exist as a single isolated compound or can be a member of a chemical (e.g., combinatorial) library. In one embodiment, the test agent is a small organic molecule. The term small organic molecules refers to molecules of a size comparable to those organic molecules generally used in pharmaceuticals. The term excludes biological macromolecules (e.g., proteins, nucleic acids, etc.). In certain embodiments, small organic molecules range in size up to about 5000 Da, up to 2000 Da, or up to about 1000 Da.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy (i.e., biopsy sample). In other embodiments, the biological sample of the present invention is a sample of bodily fluid, e.g., serum, plasma, sputum, lung aspirate, urine, and ejaculate.

The term “antibody” is meant to include intact molecules of polyclonal or monoclonal antibodies, chimeric, single chain, and humanized antibodies, as well as fragments thereof, such as Fab and F(ab′)₂, Fv and SCA fragments which are capable of binding an epitopic determinant. Monoclonal antibodies are made from antigen containing fragments of the protein by methods well known to those skilled in the art (Kohler, et al., Nature, 256:495, 1975). An Fab fragment consists of a monovalent antigen-binding fragment of an antibody molecule, and can be produced by digestion of a whole antibody molecule with the enzyme papain, to yield a fragment consisting of an intact light chain and a portion of a heavy chain. An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. An (Fab′)₂ fragment of an antibody can be obtained by treating a whole antibody molecule with the enzyme pepsin, without subsequent reduction. A (Fab′)₂ fragment is a dimer of two Fab′ fragments, held together by two disulfide bonds. An Fv fragment is defined as a genetically engineered fragment containing the variable region of a light chain and the variable region of a heavy chain expressed as two chains. A single chain antibody (“SCA”) is a genetically engineered single chain molecule containing the variable region of a light chain and the variable region of a heavy chain, linked by a suitable, flexible polypeptide linker.

Reference herein to “normal cells” or “corresponding normal cells” means cells that are from the same organ and of the same type as the cancer cell type. In one aspect, the corresponding normal cells comprise a sample of cells obtained from a healthy individual. Such corresponding normal cells can, but need not be, from an individual that is age-matched and/or of the same sex as the individual providing the cancer cells being examined. In another aspect, the corresponding normal cells comprise a sample of cells obtained from an otherwise healthy portion of tissue of a subject having hepatocellular carcinoma, nonalcoholic steatohepatitis, and/or elevated de novo lipogenesis.

Currently, there are no commonly accepted treatments that inhibit NAFLD or NASH without causing an increase in circulating lipids, a condition called hypertriglyceridemia that greatly increases the risk of cardiovascular events. By blocking the pathogenic increase in DNL, the present disclosure demonstrates the ability to prevent NAFLD and reverse NASH without causing hypertriglyceridemia. Additionally, energy expenditure is increased without a need for stimulants (such as amphetamines) or any other drugs that increase heart rate.

Progression from simple steatosis to NASH was suggested to depend on secondary hits elicited by ER stress or mitochondrial dysfunction (Farrell et al., 2012; Tilg and Moschen, 2010). Congruent with this suggestion, significantly elevated HSPA5 and ATF6 mRNA expression was detected in human NASH relative to non-fibrotic NAFLD. However, how ER stress promotes NASH progression was heretofore unknown, although it was assumed that ER stress induces cell death and inflammation through downstream mediators such as CHOP, CREBH, JNK, ASK, and NF-κB (Ji and Kaplowitz, 2006; Zhang et al., 2014).

A major metabolic process that accompanies NAFLD and NASH is de novo lipogenesis (DNL), whose rates are elevated by up to 3-fold in NAFLD/NASH patients relative to body mass index (BMI)-matched controls (Lambert et al., 2014). Elevated hepatic DNL is considered to be a primary contributor to both NAFLD and NASH and studies using tracers have established its occurrence in NAFLD patients (Paglialunga and Dehn, 2016). Such findings are also supported by free fatty acid (FFA) profiling studies (Puri et al., 2009). Similarly, HFD-fed MUP-uPA mice, which develop NASH, exhibit a large increase in their liver content of C16:O, C18:1n9, C20:3n6 and C20:4n6, FFA species that are consistent with elevated DNL (Nakagawa et al., 2014). Livers of HFD-fed MUP-uPA mice show enhanced activation of sterol regulatory element binding proteins (SREBP) (Nakagawa et al., 2014), the transcriptional regulators of sterol and FFA biosynthesis (Osborne and Espenshade, 2009). Although DNL was speculated to contribute to NASH progression by generating lipotoxic free fatty acids (FFA), recent studies suggest that most of the cytotoxicity in NASH is due to accumulation of free, non-esterified cholesterol rather than FFA (Caballero et al., 2009; Farrell and van Rooyen, 2012). Free cholesterol can induce mitochondrial dysfunction, especially in combination with TNF (Mari et al., 2006), whose expression and signaling are of importance in NASH pathogenesis (Nakagawa et al., 2014).

ER stress was also suggested to stimulate hepatic steatosis through SREBP-independent mechanisms (Zhang et al., 2014). For instance, the PERK-eIF2α-ATF4 pathway was demonstrated to increase expression of adipogenic transcription factors, including PPARγ, C/EBPα and C/EBPβ (Oyadomari et al., 2008). By contrast, the IRE1α-XBP1 pathway was shown to attenuate hepatic steatosis (Zhang et al., 2011), but in another report XBP1 was described to promote lipogenic gene induction (Lee et al., 2008), and ATF6 activation was claimed to reduce hepatic steatosis by antagonizing SREBP2 transcriptional activity (Zeng et al., 2004).

To get to the bottom of these intriguing and controversial relationships MUP-uPA mice, in which hepatocyte ER stress induced by ectopic urokinase expression (Sandgren et al., 1991) results in appearance of NASH-like pathology after high-fat diet (HFD) feeding (Nakagawa et al., 2014) were used to show that ER-stress-mediated activation of SREBP1 and 2, TG and cholesterol accumulation, as well as NASH progression are entirely dependent on Casp2, whose expression is ER-stress-inducible.

Although MUP-uPA mice kept on normal chow (NC) show transient liver damage and steatosis within 4-6 weeks after birth, this damage subsides due to liver repopulation with hepatocytes in which uPA expression is reduced due to transgene methylation (Weglarz et al., 2000). Nonetheless, HFD feeding re-ignites ER stress, resulting in appearance of classical NASH signs within 16 weeks or less (Nakagawa et al., 2014). NASH development remains ER-stress-dependent and is effectively inhibited by chemical chaperons, such as phenyl butyric acid (PBA) and tauroursodeoxycholic acid (TUDCA), or Bip/GRP78 overexpression (Nakagawa et al., 2014). Importantly, these treatments inhibit lipid accumulation and attenuate activation of sterol response element binding proteins (SREBP), whose activity is higher in MUP-uPA mice than in non-transgenic controls (Nakagawa et al 2014). These results suggest that the SREBP1 and 2, which activate transcriptional programs that promote FA and cholesterol synthesis, respectively (Osborne and Espenshade, 2009), may play a key role in NAFLD/NASH pathogenesis. Given the NASH-specific accumulation of hepatic cholesterol (Caballero et al., 2009; Puri et al., 2007), it is plausible that the mysterious switch from bland steatosis to NASH depends on SREBP2, whose expression was reported to be considerably higher in NASH than in early non-fibrotic NAFLD (Caballero et al., 2009). Curiously, hepatitis C virus infection is also associated with SREBP activation and hepatic steatosis (Pekow et al., 2007; Waris et al., 2007).

SREBP1 isoforms (liver mainly expresses SREBP1c) and SREBP2 are produced as inactive precursors that are embedded in the endoplasmic reticulum (ER) membrane, together with the INSIG:SCAP chaperone complex. Of the two, regulation of SREBP2, which controls sterol biosynthesis, is better understood, especially in respect to its negative feedback inhibition by cholesterol (Brown and Goldstein, 1997a). Cholesterol deficiency disrupts the INSIG:SCAP complex and results in translocation of SCAP-bound SREBP2 to the early Golgi apparatus where it is processed by the site specific proteases, SIP and S2P, which release its cytoplasmic-facing N-terminal portion to enter the nucleus to stimulate transcription of sterol biosynthetic genes (Osborne and Espenshade, 2009). SREBP1 is also produced as an inactive ER-anchored precursor that interacts with the INSIG:SCAP complex, but the cues that trigger its proteolytic activation by S1P and S2P are complex and include insulin, oxysterols and feeding cues (Browning and Horton, 2004; Owen et al., 2012).

Despite the well-established negative feedback control of SREBP activation, HFD consumption promotes activation of SREBP1 and 2 in both WT and MUP-uPA mice (Nakagawa et al., 2014). ER stress also induces SREBP1 and 2 activation in cultured cells, but the underlying mechanisms are far from clear (Colgan et al., 2011). One proposed mechanism involves INSIG downregulation due to PERK-mediated eIF2a phosphorylation and translational inhibition (Bobrovnikova-Marj on et al., 2008; Lee and Ye, 2004).

Caspases are a family of cytosolic aspartate-specific cysteine proteases involved in the initiation and execution of apoptosis. They are expressed as latent zymogens and are activated by an autoproteolytic mechanism or by processing other proteases (frequently other caspases). Human caspases can be subdivided into three functional groups: cytokine activation (caspase-1, -4, -5, and -13), apoptosis initiation (caspase-2, -8, -9, and -10), and apoptosis execution (caspase-3, -6, and -7). Caspases are regulated by a variety of stimili, including APAF1, CFLAR/FLIP, NOL3/ARC, and members of the inhibitor of apoptosis (IAP) family such as BIRC1/NAIP, BIRC2/cIAP-1, BIRC3/cIAP-2, BIRC4/XIAP, BIRC5/Survivin, and BIRC7/Livin.

Another proposal is caspase-mediated SREBP processing (Colgan et al., 2011). Activated caspase-3 (Casp3) can elicit S1P/S2P-independent SREBP activation (Wang et al., 1996) and other studies have implicated TNF and caspases-4 and -12 (Casp4, Casp12) in SREBP activation in alcohol-exposed cells, operating via a cholesterol-insensitive mechanism (Pastorino and Shulga, 2008). Most caspases are activated during apoptosis, but hepatocytes in which DNL and cholesterol synthesis are persistently engaged are not apoptotic and only undergo ballooning degeneration and cell death after excessive lipid droplet buildup, TNF exposure, and extensive reactive oxygen species (ROS) generation (Nakagawa et al., 2014).

Inhibition of TNF signaling attenuates lipid droplet accumulation and ballooning degeneration and prevents NASH progression in HFD-fed MUP-uPA mice. Accordingly, it was postulated that a non-apoptotic caspase whose activity or expression is induced by ER stress or inflammation/TNF may control SREBP activation, DNL, and hepatic cholesterol accumulation during NASH development in MUP-uPA mice and human patients.

One such caspase is caspase-2 (Casp2), which was shown to be upregulated and activated during ER stress (Upton et al., 2012) and on SREBP2 activation (Logette et al., 2005). Unlike other caspases, Casp2 has been detected in the ER lumen and the Golgi apparatus, results that further support a link between ER stress and Casp2. Casp2 is composed of protein-protein interaction domain, CARD, and two small catalytic subunits in where catalytic dyad is composed of cysteine and histidine. Due to its structural similarity to caspase 8, a primary initiator of proteolytic cascades that induce programmed cell death, Casp2 is currently thought to be a cell death inducer. Correspondingly, the majority of previous experiments have been focused on the role of Casp2 in cell death, using fibroblasts, B cells, T cells and neuroblastoma cells.

Although Casp2 has been implicated in ER stress-induced cell death (Upton et al., 2012), this particular function has been questioned (Lu et al., 2014; Sandow et al., 2014). Furthermore, knockout mouse studies have failed to identify a clear role for Casp2 in any known and physiologically essential apoptotic process, suggesting that Casp2 may be a non-apoptotic caspase (Bouchier-Hayes and Green, 2012; Kumar, 2009; Fava 2012; each of which is incorporated herein by reference). Nonetheless, more recent studies implicated Casp2 in induction of liver damage and lipotoxicity in mice fed with a toxic diet deficient in methionine and choline (MCD) (Machado et al., 2015). Although MCD was used to model NASH, the extensive liver damage, weight loss, and lethality induced by this diet make it an unsuitable surrogate for the human condition, which is linked to weight gain and is not associated with acute liver failure (Ibrahim et al., 2016; Larter and Yeh, 2008).

These earlier studies had not used an appropriate model of NASH and had therefore concluded that Casp2 activation promotes cholestatic liver injury and lipotoxicity due to its apoptotic activity and had not examined the role of Casp2 in the control of DNL, cholesterol accumulation or SREBP activation. For example, after being fed with MCD for 4 to 6 weeks, mice exhibit severe hepatic lipid accumulation and inflammation, but such mice do not gain weight. In fact, they lose weight due to liver failure. Therefore, metabolic complications and hyperlipidemia that are caused by central obesity are absent in these mice. HFD-fed ob/ob and db/db mice become very fat due to uncontrolled eating behavior, caused by the absence of leptin signal. However, such mice do not show liver hepatic damage or inflammation, indicating that they do not develop NASH. Lastly, upon HFD feeding, foz/foz mice exhibit severe obesity accompanied with insulin resistance, altered lipid profiles, hepatic damage, and inflammation. However, appearances of these metabolic and pathogenic features are highly dependent the on genetic background of the mice and are not always reproducible.

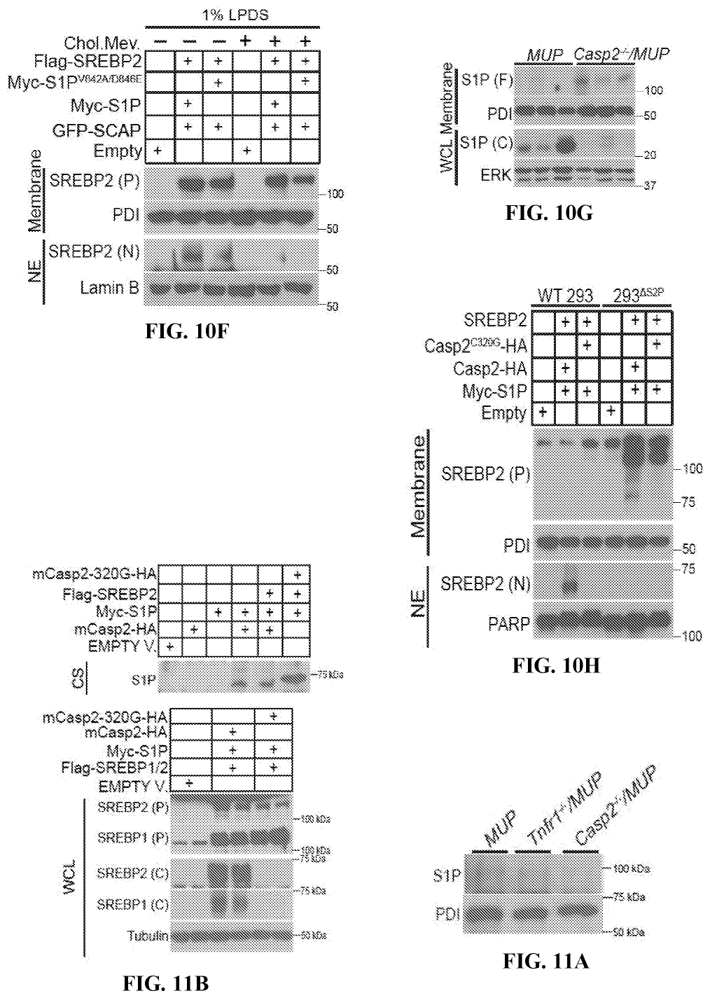

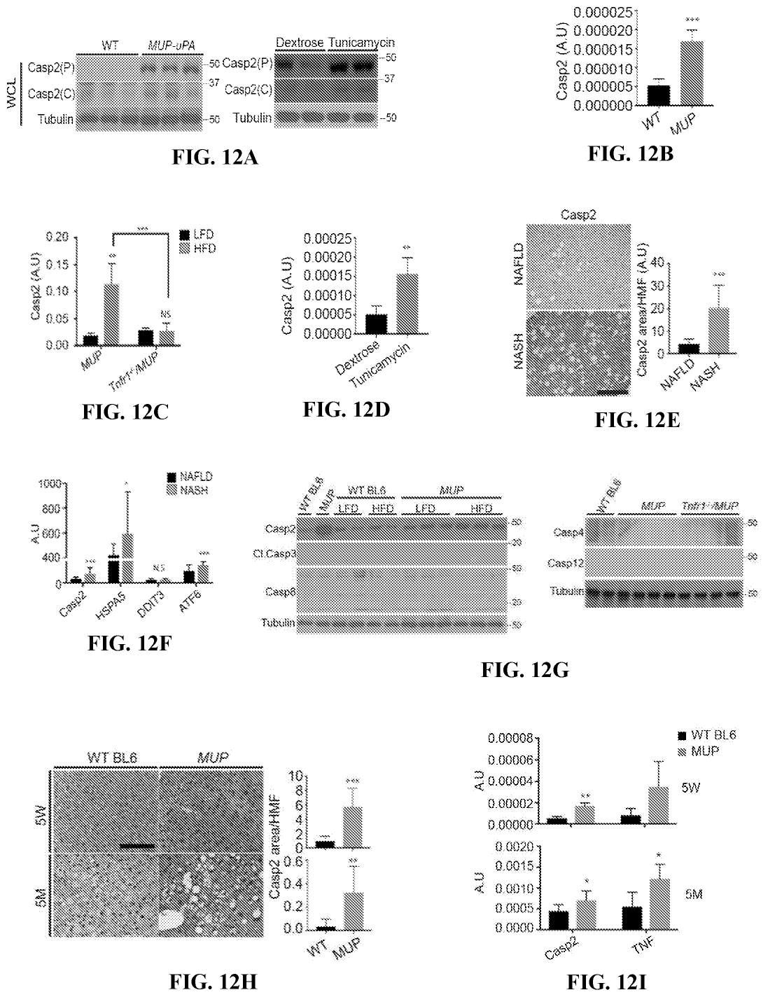

To overcome such technical limitations, a new mouse model for studying NASH was developed. MUP-uPA mice, in which excessive urokinase plasminogen activator (uPA) synthesis, express uPA exclusively in the liver resulting in induction of ER stress, which goes away after 6-7 weeks of age. However, after feeding with HFD, ER stress is restored and, in combination with liver steatosis, it leads to fatty liver inflammation that is identical in histopathological and biochemical features to human NASH. Furthermore, HFD-fed MUP-uPA mice progress to liver fibrosis, indicating that HFD-fed MUP-uPA mice are a proper mouse model for studying both NASH and NASH-induced HCC. It was found that MUP-uPA mice exhibit upregulation of the precursor and the cleaved (active) forms of Casp2 at 5 weeks of age, a time at which profound SREBP activation is observed (FIG. 12A). Importantly, Casp2 is also highly expressed in the liver of patients with NASH but not in NAFLD patients (FIG. 12B), and was instrumental for hepatic TG and cholesterol accumulation and NASH progression in mice.

Accordingly, the data presented herein shows that Casp2 is a critical regulator of DNL during NASH progression and that either its ablation or pharmacological inhibition prevent SREBP activation, DNL, hepatic steatosis, inflammation and other NASH-related symptoms. In fact, the effect of Casp2 inhibition is at least as strong as the effect of chemical or biological chaperons, previously found to ameliorate liver ER stress and inhibit NASH development (Nakagawa et al., 2014).

Importantly, the main conduit through which Casp2 affects NASH development is SIP, whose activation by Casp2 results in dysregulated SREBP activation and signaling, leading to TG and cholesterol accumulation within hepatocytes. Thus, the data presented herein also shows that Casp2 can directly activate SIP, one of whose proteolytic fragments is secreted to the serum of HFD-fed MUP-uPA mice and human NASH patients, providing a much needed biomarker for non-invasive monitoring of NASH progression and severity. The same biomarker can be used to monitor Casp2 activity before and after drug treatment. Given that Casp2 expression is elevated in NASH-afflicted human liver, these results support the notion that Casp2 activation plays a critical role in human NASH pathogenesis.

Therefore, Casp2 activation leads to excessive lipid synthesis and surplus lipids incorporate into lipid droplets, resulting in lipid droplet hypertrophy that is directly linked to NASH progression. The present invention therefore provides for the inhibition of this process by targeting Casp2, thereby preventing NASH development and attenuating central obesity by reducing lipogenesis not only in adipocytes but also in adipose tissue. In addition, the present invention allows the conversion of obese adipocytes (white visceral fat) to a healthier phenotype called “beige” adipocytes. This effect also correlates with inhibition of lipogenesis. All of these effects, enable Casp2 targeting drugs to inhibit the development of NAFLD and NASH, and reduce the severity of NASH in animals in which NASH has already been established.

In addition to NASH, Casp2 regulates energy expenditure. Casp2-deficient mice consume as much food as Casp2-proficient mice but do not gain weight and show elevated O₂ consumption and CO₂ production. Enhanced energy expenditure is accompanied by a marked increase in AMPK phosphorylation in all metabolically active tissues, suggesting that although initial ER stress in MUP-uPA mice is liver specific, elicited by uPA expression, Casp2 controls energy expenditure also in fat and muscle. Indeed, adipocytes in HFD-fed Casp2^{-/-} mice are much less hypertrophic than WT adipocytes and are not surrounded by a crown of adipose tissue macrophages. It is not clear whether all of these effects are due to inhibition of SREBP activation, but upregulation of DNL shunts large amount of acetyl-CoA into FA and TG synthesis (Sanders and Griffin, 2016). Downregulation of DNL in Casp2-deficient mice prevents energy storage and enhances energy expenditure and thermogenesis. Curiously, however, SREBP1 ablation in leptin-deficient ob/ob mice prevents hepatic steatosis but not obesity (Yahagi et al., 2002). Conversely, transgenic expression of activated SREBP1c results in hepatic steatosis, whereas activation of AMPK inhibits SREBP1 activation (Horton et al., 2002).

Treatment of HFD-fed MUP-uPA mice with a Casp2 inhibitor prevents NASH progression and liver damage as well as hepatic steatosis, TG and cholesterol accumulation. Casp2 inhibition causes AMPK activation in liver and muscle and prevents adipose tissue hypertrophy and inflammation. These results further indicate that in contrast to a previous study in which extensive liver damage was induced by consumption of the toxic MCD diet (Machado et al., 2015), Casp2 does not exert its NASH-promoting effect by eliciting lipotoxic cell death. Instead, Casp2 acts as the critical pathological regulator of DNL, cholesterol metabolism and energy expenditure, in part via its ability to activate SREBP1 and 2.

IDN-6556 is a small molecule, broad-spectrum caspase inhibitor (pan-caspase inhibitor) with activity against all tested human caspases (including caspase-2). IDN-6556 shows no inhibition of other classes of proteases or other enzymes or receptors other than caspases (Idun Pharmaceuticals.) VDVAD is a cell-permeable, synthetic peptide that irreversibly inhibits the activity of caspase-2. It is available as a fluoromethylketone derivative that facilitates inhibition of cysteine proteases in a caspase-2-specific manner. A number of companies sell variations of this peptide inhibitor (for example, R&D Systems). Monoclonal antibodies specific for caspase-2 are available (e.g., clone 691233; R&D Systems). DARPin (i.e., Designed Ankyrin Repeat Proteins) is a genetically engineered antibody mimetic that has been described for caspase-2. (Schweizer, et al. 2007). Thus, exemplary inhibitors of caspase-2 include, but are not limited to, Ac-VDVAD-CHO (C₂₃H₃₇N₅O₁₀), z-VDVAD-FMK (C₃₂H₄₆FN₅O₁₁), and Z-FA-FMK (C₂₁H₂₃FN₂O₄). See US Pub. Nos. 2012/0196892 and 2016/0331801, incorporated herein by reference.

In various embodiments, the inhibitor of caspase-2 is an inhibitory nucleic acid that specifically inhibits expression of casp2 and/or inhibits casp2 synthesis. As used herein, an “inhibitory nucleic acid” means an RNA, DNA, or a combination thereof that interferes or interrupts the translation of mRNA. Inhibitory nucleic acids can be single or double stranded. The nucleotides of the inhibitory nucleic acid can be chemically modified, natural or artificial. The terms “short-inhibitory RNA” and “siRNA” interchangeably refer to short double-stranded RNA oligonucleotides that mediate RNA interference (also referred to as “RNA-mediated interference” or “RNAi”). The terms “small hairpin RNA” and “shRNA” interchangeably refer to an artificial RNA molecule with a tight hairpin turn that can be used to silence target gene expression via RNAi. RNAi is a highly conserved gene silencing event functioning through targeted destruction of individual mRNA by a homologous double-stranded small interfering RNA (siRNA) (Fire, A. et al., Nature 391:806-811 (1998)). Mechanisms for RNAi are reviewed, for example, in Bayne and Allshire, Trends in Genetics (2005) 21:370-73; Morris, Cell Mol Life Sci (2005) 62:3057-66; Filipowicz, et al., Current Opinion in Structural Biology (2005) 15:331-41.

Methods for the design of siRNA or shRNA target sequences have been described in the art. Among the factors to be considered include: siRNA target sequences should be specific to the gene of interest and have about 20-50% GC content (Henshel et al., Nucl. Acids Res., 32: 113-20 (2004); G/C at the 5′ end of the sense strand; A/U at the 5′ end of the antisense strand; at least 5 A/U residues in the first 7 bases of the 5′ terminal of the antisense strand; and no runs of more than 9 G/C residues (Ui-Tei et al., Nucl. Acids Res., 3: 936-48 (2004)). Additionally, primer design rules specific to the RNA polymerase will apply. For example, for RNA polymerase III, the polymerase that transcribes from the U6 promoter, the preferred target sequence is 5′-GN18-3′. Runs of 4 or more Ts (or As on the other strand) will serve as terminator sequences for RNA polymerase III and should be avoided. In addition, regions with a run of any single base should be avoided (Czauderna et al., Nucl. Acids Res., 31: 2705-16 (2003)). It has also been generally recommended that the mRNA target site be at least 50-200 bases downstream of the start codon (Sui et al., Proc. Natl. Acad. Sci. USA, 99: 5515-20 (2002); Elbashir et al., Methods, 26: 199-213 (2002); Duxbury and Whang, J. Surg. Res., 117: 339-44 (2004) to avoid regions in which regulatory proteins might bind. Additionally, a number of computer programs are available to aid in the design of suitable siRNA and shRNAs for use in suppressing expression of casp2 and/or inhibiting casp2 synthesis.

Ribozymes that cleave mRNA at site-specific recognition sequences can be used to destroy target mRNAs, particularly through the use of hammerhead ribozymes. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art.

Gene targeting ribozymes may contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA.

With regard to antisense, siRNA or ribozyme oligonucleotides, phosphorothioate oligonucleotides can be used. Modifications of the phosphodiester linkage as well as of the heterocycle or the sugar may provide an increase in efficiency. Phophorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with elements including a Cas gene and specifically designed CRISPRs, nucleic acid sequences can be cut and modified at any desired location. Methods of preparing compositions for use in genome editing using the CRISPR/Cas systems are described in detail in US Pub. No. 2016/0340661, US Pub. No. 20160340662, US Pub. No. 2016/0354487, US Pub. No. 2016/0355796, US Pub. No. 20160355797, and WO 2014/018423, which are specifically incorporated by reference herein in their entireties.

Thus, as used herein, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer”, “guide RNA” or “gRNA” in the context of an endogenous CRISPR system), or other sequences and transcripts from a CRISPR locus. One or more tracr mate sequences operably linked to a guide sequence (e.g., direct repeat-spacer-direct repeat) can also be referred to as “pre-crRNA” (pre-CRISPR RNA) before processing or crRNA after processing by a nuclease.

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr, a tool designed to help scientists find CRISPR targeting sites in a wide range of species and generate the appropriate crRNA sequences.

Accordingly, the present invention provides a method of treating nonalcoholic steatohepatitis (NASH), nonalcoholic fatty liver disease (NAFLD), and/or elevated de novo lipogenesis (DNL) in a subject in need thereof. The method includes administering to the subject an effective amount of an inhibitor of caspase-2 activity or expression. In various embodiments, the method may further include measuring the expression or activity of caspase-2 a cell sample of the subject to be treated, and determining that caspase-2 activity or expression is decreased after administration of the inhibitor, as compared to the level of caspase-2 activity or expression prior to administration of the inhibitor. Such a detected decrease confirms treatment of NASH, NAFLD, and/or elevated DNL in the subject.

As indicated above, the inhibitor of caspase-2 activity or expression and/or casp2 synthesis may be an inhibitory nucleic acid that is administered to the subject. Inhibitory nucleic acids, such as siRNA, shRNA, ribozymes, or antisense molecules, can be synthesized and introduced into cells using methods known in the art. Molecules can be synthesized chemically or enzymatically in vitro (Micura, Agnes Chem. Int. Ed. Emgl. 41: 2265-9 (2002); Paddison et al., Proc. Natl. Acad. Sci. USA, 99: 1443-8 2002) or endogenously expressed inside the cells in the form of shRNAs (Yu et al., Proc. Natl. Acad. Sci. USA, 99: 6047-52 (2002); McManus et al., RNA 8, 842-50 (2002)). Plasmid-based expression systems using RNA polymerase III U6 or H1, or RNA polymerase II U1, small nuclear RNA promoters, have been used for endogenous expression of shRNAs (Brummelkamp et al., Science, 296: 550-3 (2002); Sui et al., Proc. Natl. Acad. Sci. USA, 99: 5515-20 (2002); Novarino et al., J. Neurosci., 24: 5322-30 (2004)). Synthetic siRNAs can be delivered by electroporation or by using lipophilic agents (McManus et al., RNA 8, 842-50 (2002); Kishida et al., J. Gene Med., 6: 105-10 (2004)). Alternatively, plasmid systems can be used to stably express small hairpin RNAs (shRNA) for the suppression of target genes (Dykxhoorn et al., Nat. Rev. Mol. Biol., 4: 457-67 (2003)). Various viral delivery systems have been developed to deliver shRNA-expressing cassettes into cells that are difficult to transfect (Brummelkamp et al., Cancer Cell, 2: 243-7 (2002); Rubinson et al., Nat. Genet., 33: 401-6 2003). Furthermore, siRNAs can also be delivered into live animals. (Hasuwa et al., FEBS Lett., 532, 227-30 (2002); Carmell et al., Nat. Struct. Biol., 10: 91-2 (2003); Kobayashi et al., J. Pharmacol. Exp. Ther., 308: 688-93 (2004)).

Inhibitory oligonucleotides can be delivered to a cell by direct transfection or transfection and expression via an expression vector. Appropriate expression vectors include mammalian expression vectors and viral vectors, into which has been cloned an inhibitory oligonucleotide with the appropriate regulatory sequences including a promoter to result in expression of the antisense RNA in a host cell. Suitable promoters can be constitutive or development-specific promoters. Transfection delivery can be achieved by liposomal transfection reagents, known in the art (e.g., Xtreme transfection reagent, Roche, Alameda, Calif.; Lipofectamine formulations, Invitrogen, Carlsbad, Calif.). Delivery mediated by cationic liposomes, by retroviral vectors and direct delivery are efficient. Another possible delivery mode is targeting using antibody to cell surface markers for the target cells.

In some embodiments, one or more vectors driving expression of one or more elements of a CRISPR system are introduced into a target cell such that expression of the elements of the CRISPR system direct formation of a CRISPR complex at one or more target sites. Accordingly, cleavage of DNA by the genome editing vector or composition can be used to delete nucleic acid material from a target DNA sequence by cleaving the target DNA sequence and allowing the cell to repair the sequence. As such, the compositions can be used to modify DNA in a site-specific, i.e., “targeted” way, for example gene knock-out, gene knock-in, gene editing, gene tagging, etc., as used in, for example, gene therapy.

While the specifics can be varied in different engineered CRISPR systems, the overall methodology is similar. A practitioner interested in using CRISPR technology to target a DNA sequence can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

In another aspect, the present invention provides a method of ameliorating NASH, NAFLD, elevated DNL, and/or NASH-induced hepatocellular carcinoma in a subject. As used herein, the term “ameliorate” means that the clinical signs and/or the symptoms associated with NASH, NAFLD, DNL and/or NASH-induced hepatocellular carcinoma are lessened. The signs or symptoms to be monitored will be characteristic of hepatocellular carcinoma, NASH, NAFLD, DNL and/or NASH-induced hepatocellular carcinoma and will be well known to the skilled clinician, as will the methods for monitoring the signs and conditions.

An agent useful in a method of the invention can be any type of molecule, for example, a polynucleotide, a peptide, a peptidomimetic, peptoids such as vinylogous peptoids, a small organic molecule, or the like, and can act in any of various ways to reduce or inhibit elevated caspase-2 activity or expression. Further, the agent can be administered in any way typical of an agent used to treat the particular type of hepatocellular carcinoma, nonalcoholic steatohepatitis, and/or de novo lipogenesis or under conditions that facilitate contact of the agent with the target cancer cells and, if appropriate, entry into the cells. Entry of a polynucleotide agent into a cell, for example, can be facilitated by incorporating the polynucleotide into a viral vector that can infect the cells. If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell.

Generally, the agent is formulated in a composition (e.g., a pharmaceutical composition) suitable for administration to the subject, which can be any vertebrate subject, including a mammalian subject (e.g., a human subject). Such formulated agents are useful as medicaments for treating a subject suffering from hepatocellular carcinoma, nonalcoholic steatohepatitis, and/or elevated DNL that are characterized, in part, by elevated or abnormally elevated caspase-2 activity or expression, or abnormal SREBP activation of SP cleavage.

Pharmaceutically acceptable carriers useful for formulating an agent for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day which can be administered in single or multiple doses.

The invention also provides a method of determining whether NASH, NAFLD or NASH-induced hepatocellular carcinoma in a given subject is amenable to treatment with an inhibitor of caspase-2 as disclosed herein. The method can be performed, for example, by measuring the expression or activity of caspase-2 a cell sample or serum sample of a subject to be treated, and determining that caspase-2 activity or expression is elevated or abnormally elevated as compared to the level of caspase-2 activity or expression in corresponding normal cells or control serum, which can be a sample of normal (i.e., not cancer) cells of the subject having hepatocellular carcinoma. Detection of elevated or abnormally elevated level of caspase-2 activity or expression in the cells as compared to the corresponding normal cells indicates that the subject can benefit from treatment with an inhibitor of caspase-2. A sample of cells used in the present method can be obtained using a biopsy procedure (e.g., a needle biopsy), or can be a sample of cells obtained by a surgical procedure to remove and/or debulk the tumor.

The method of identifying NASH, NAFLD or NASH-induced hepatocellular carcinoma amenable to treatment with a an inhibitor of caspase-2 can further include contacting cells of the sample with at least one test agent, and detecting a decrease in caspase-2 activity or expression in the cells following said contact. Such a method provides a means to confirm that the NASH, NAFLD or hepatocellular carcinoma is amenable to treatment with an inhibitor of caspase-2. Further, the method can include testing one or more different test agents, either alone or in combination, thus providing a means to identify one or more test agents useful for treating the particular NASH, NAFLD or hepatocellular carcinoma cells being examined. Accordingly, the present invention also provides a method of identifying an agent useful for treating NASH, NAFLD or hepatocellular carcinoma in a subject.

In another aspect, the invention provides a method of detecting NASH in a subject and/or confirming a diagnosis of NASH in a subject. The method includes detecting cleaved and secreted S1P in a serum sample from the subject, in addition to known methods of detecting and/or diagnosing NASH in a subject. Detection of cleaved and secreted S1P in the serum sample of the subject is indicative of NASH and/or progression towards NASH in the subject.

In another aspect, the present invention provides a method of identifying an agent useful for treating hepatocellular carcinoma, NASH, NAFLD, and/or elevated DNL. The method includes contacting a sample of cells with at least one test agent, wherein a decrease in caspase-2 activity or expression in the presence of the test agent as compared to caspase-2 activity or expression in the absence of the test agent identifies the agent as useful for treating hepatocellular carcinoma, NASH, NAFLD, and/or elevated DNL. Thus, the invention likewise provides a method of screening for casp2 inhibitors.

When practiced as an in vitro assay, the methods can be adapted to a high throughput format, thus allowing the examination of a plurality (i.e., 2, 3, 4, or more) of cell samples and/or test agents, which independently can be the same or different, in parallel. A high throughput format provides numerous advantages, including that test agents can be tested on several samples of cells from a single patient, thus allowing, for example, for the identification of a particularly effective concentration of an agent to be administered to the subject, or for the identification of a particularly effective agent to be administered to the subject. Alternatively or in addition thereto, the high throughput format may be used to screen for casp2 inhibitors using an SREBP1/2-dependent report in cells transfected with casp2 with or without SIP expression vectors.

As such, a high throughput format allows for the examination of two, three, four, etc., different test agents, alone or in combination, on the hepatocellular carcinoma or NASH cells of a subject such that the best (most effective) agent or combination of agents can be used for a therapeutic procedure. Further, a high throughput format allows, for example, control samples (positive controls and or negative controls) to be run in parallel with test samples, including, for example, samples of cells known to be effectively treated with an agent being tested.

A high throughput method of the invention can be practiced in any of a variety of ways. For example, different samples of cells obtained from different subjects can be examined, in parallel, with same or different amounts of one or a plurality of test agent(s); or two or more samples of cells obtained from one subject can be examined with same or different amounts of one or a plurality of test agent. In addition, cell samples, which can be of the same or different subjects, can be examined using combinations of test agents and/or known effective agents. Variations of these exemplified formats also can be used to identify an agent or combination of agents useful for treating hepatocellular carcinoma having elevated caspase-2 activity or expression.

When performed in a high throughput (or ultra-high throughput) format, the method can be performed on a solid support (e.g., a microtiter plate, a silicon wafer, or a glass slide), wherein samples to be contacted with a test agent are positioned such that each is delineated from each other (e.g., in wells). Any number of samples (e.g., 96, 1024, 10,000, 100,000, or more) can be examined in parallel using such a method, depending on the particular support used. Where samples are positioned in an array (i.e., a defined pattern), each sample in the array can be defined by its position (e.g., using an x-y axis), thus providing an “address” for each sample. An advantage of using an addressable array format is that the method can be automated, in whole or in part, such that cell samples, reagents, test agents, and the like, can be dispensed to (or removed from) specified positions at desired times, and samples (or aliquots) can be monitored, for example, for caspase-2 activity or expression and/or cell viability.

The following examples are intended to illustrate but not limit the invention.

### Example 1

### Materials and Methods

Casp2^{-/-} mice were purchased from The Jackson Laboratories and crossed with either C57BL/6 or MUP-uPA mice (Sandgren et al., 1991; Weglarz et al., 2000) to generate Casp2^{-/-}/WT or Casp2^{-/-}/MUP-uPA mice, respectively. All mouse lines were either on a pure C₅₇BL/6 genetic background or crossed into it for at least nine generations. Mice were fed with HFD (#S3282, Bio-serv) for a total of 12 weeks, starting at 8 weeks of age. Body weight increase and food consumption were monitored bi-weekly throughout the entire feeding period. Mice were starved for 3 hr before sacrifice and liver and adipose tissue were excised and weighed.

Generation of Cell Lines—

SCAP, S1P, or S2P knock-out HEK293 cells (293^{ΔSCAP}, 293^{ΔS1P}, or 293^{ΔS2P}) were generated using LentiCRISPRv2 system (Sanjana et al., 2014). Two oligonucleotides that contain guide sequences against SCAP, SIP, or S2P were synthesized and annealed to lentiCRISPRv2 vector. Insertion of oligonucleotides into vector was verified by DNA sequencing. Guide sequences were delivered into HEK293 cells through viral transduction and SCAP, S1P, or S2P-deleted 293 cells (293^{ΔSCAP}, 293^{ΔS1P} or 293^{ΔS2P}) were selected in DMEM/F12 medium supplemented with puromycin (10 μg/ml). Cholesterol and lipid auxotrophic 293^{ΔSCAP} 293^{ΔS1P} or 293^{ΔS2P} cells were maintained in medium supplemented with 10% FBS, 5 μg/ml cholesterol, 20 μM sodium oleate, and 1 mM mevalonate. Following oligonucleotides were used for deletion of SCAP, SIP, or S2P in HEK293 cells:

MBTPS1-F:
(SEQ ID NO: 8)
5′-CAC CGG AAC GAA AGA CTT TTC GTT G-3′;
MBTPS1-R:
(SEQ ID NO: 9)
5′-AAA CCA ACG AAA AGT CTT TCG TTC C-3′;
MBTPS2-F:
(SEQ ID NO: 10)
5′-CAC CGC CGC CGT CCC CAA CTG TAA A-3′;
MBTPS2-R:
(SEQ ID NO: 11)
5′-AAA CTT TAC AGT TGG GGA CGG CGG C-3′;
SCAP-F:
(SEQ ID NO: 12)
5′-CAC CGG CTG CGT GAG AAG ATA TCT C-3′;
SCAP-R:
(SEQ ID NO: 13)
5′-AAA CGA GAT ATC TTC TCA CGC AGC C-3′.

Reagents—

Cholesterol was obtained from Santa Cruz Technologies (#sc-202539). Sodium oleate (#07501), (R)-(−)-mevalonolactone (#68519), and Ac-VDVAD-CHO (#SCP0076) were from Sigma-Aldrich. Total Exosome Isolation Reagent for both plasma (#4484450) and cell culture media (#4478359) were from Thermo Fisher Scientific. PNGase F (#P0704) was from New England BioLabs. Antibodies used for IB analysis were: anti-Casp2 for IB analysis (#ALX-804-356, Enzo Life Sciences), anti-Casp2 for IHC (#Ab2251, Abcam), anti-p62 (#GP62-C, ProGen), anti-SREBP1 (#Ab3259, Abcam), anti-SREBP2 (#Ab30682, Abcam), anti-S1P for detection of N-terminal fragments (#sc-271916, Santa Cruz Technologies), anti-S1P for detection of the catalytic pocket (#Ab140592, Abcam), anti-phospho-AMPK (#2535, Cell Signaling Technologies), anti-AMPK (#sc-25792, Santa Cruz Technologies), anti-ERK (#9102, Cell Signaling Technologies), anti-ACC (#3662, Cell Signaling Technologies), anti-FAS (#3180, Cell Signaling Technologies), anti-Hmgcr (#sc-33827, Santa Cruz Technologies), anti-Hmgcs (#sc-373681, Santa Cruz Technologies), PARP (#sc-7150, Santa Cruz Technologies), anti-UCP1 (#10983, Abcam), anti-F4/80 (#MF48000, Thermo Fisher Scientific), anti-HA (#1867431, Roche), anti-Myc (#05-724, Upstate), anti-Flag (#F7425, Sigma-Aldrich) and anti-tubulin (#T5168, Sigma-Aldrich).

Transfections and Cell Fractionation—

To test SP processing, cells were plated at a density of 5×10⁵ per well of 6 well plates. On the next day, 1 μg of indicated vector DNAs were transfected using Lipofectamine 3000 (Thermo Fisher Scientific, MA) according to manufacturer's instruction. After 5 hr, cells were incubated in DMEM/F12 medium supplemented with 1% lipoprotein deficient serum (LPDS). For SREBP cleavage, cells were plated on 5×10 cm plates at a density of 4×10⁶ per plate. The next day, cells were transfected with 5 μg of each plasmid DNA using Lipofectamine 3000 (Thermo Fisher Scientific, MA) according to manufacturer's instructions and incubated in DMEM/F12 medium supplemented with 1% LPDS for 24 hr. Nuclear and membrane fractions were prepared as described previously (DeBose-Boyd et al., 1999). Briefly, cells were incubated in hypotonic buffer A (10 mM HEPES-KOH, pH 7.6, 10 mM KCl, 1.5 mM MgCl², 1 mM sodium EDTA, 1 mM sodium EGTA, 250 mM sucrose, and 5 g/ml pepstatin A, 10 g/ml leupeptin, 0.5 mM PMSF, 1 mM DTT, and 25 g/ml ALLN) for 1 hr and passed through a 26G-gauge needle 30 times. Cell lysates were centrifuged at 890 g at 4° C. for 10 min to collect nuclei. Pellets were resuspended in lysis buffer (150 mM Tris-HCl, pH 7.4, 10% sodium-deoxycholate, 100 mM NaCl, 100 mM EDTA, 100 mM PMSF, 200 mM NaF, 100 mM Na₃VO₄, and a mixture of protease inhibitors) to collect nuclear extracts. The supernatants from the original 890 g spin were centrifuged at 60,000 g for 1 hr at 4° C. in a Beckman centrifuge (TLA 100.3 rotor) to collect membranes. Pellets were subjected to sonication and removed of carbohydrate moieties by PNGase F. Prepared nuclear and membrane extracts were subjected to IB analysis.

Detection of Secreted S1P—

50 μl of plasma was incubated with 10 μl of exosome isolation slurry for 30 min at 4° C. After centrifugation, the sedimented pellet was resuspended in PBS, followed by sonication and incubation with PNGase F. 10 μl of plasma samples were applied to IB analysis with S1P antibody. For detection of secreted S1P, culture supernatants were mixed with exosome isolation slurry and left at 4° C. overnight. After centrifugation, pellets were resuspended, subjected to sonication, and carbohydrate moieties were removed. SP was detected by IB analysis.

RNA Analysis—

RNAs were extracted from liver or fat tissue of indicated mice or human biopsies using TRIZOL reagent according to manufacturer's instruction. cDNAs were synthesized from total RNAs using Super Script VILO cDNA Synthesis Kit (Thermo Fisher Scientific, MA) according to the supplier's instructions. The cDNAs were quantified by real-time PCR analysis using SYBR Green Master Mix (Bio-Rad, CA). Primer sequences that were used for human biopsies are as follows:

Casp2-F:
(SEQ ID NO: 14)
5′-CTA CAT GAC CAG ACC GCA CA-3′;
Casp2-R:
(SEQ ID NO: 15)
5′-GTG CCA CTA CGC AGG AGT G-3′;
DDIT3-F:
(SEQ ID NO: 16)
5′-AGC CAA AAT CAG AGC TGG AA-3′;
DDIT3-R:
(SEQ ID NO: 17)
5′-TGG ATC AGT CTG GAA AAG CA-3′;
ATF6-F:
(SEQ ID NO: 18)
5′-GCA GAA GGG GAG ACA CAT TT-3′;
ATF6-R:
(SEQ ID NO: 19)
5′-TTG ACA TTT TTG GTC TTG TGG-3′;
HSPA5-F:
(SEQ ID NO: 20)
5′ CAC AGT GGT GCC TAC CAA GA-3′;
HSPA5-R:
(SEQ ID NO: 21)
5′-TGT CTT TTG TCA GGG GTC TTT-3′;
PRL32-F:
(SEQ ID NO: 22)
5′-TGT CGC AGA GTG TCT TCC AA-3′;
PRL32-R:
(SEQ ID NO: 23)
5′-CCG TCC CTT CTC TCT TCC TC-3′.

The following primers were used to determine mRNA in fat or liver of mice:

Casp2-F:
(SEQ ID NO: 24)
5′-CAC CCT CTT CAA GCT TTT GG-3′;
Casp2-R:
(SEQ ID NO: 25)
5′-CGA AAA ACC TCT TGG AGC TG-3′;
uPA-F:
(SEQ ID NO: 26)
5′-CTT CCC ACT ACC TTG GCT GG-3′;
uPA-R:
(SEQ ID NO: 27)
5′-CCA GCC AAG GTA GTG GGA AG-3′;
TNFα-F:
(SEQ ID NO: 28)
5′-ACG GCA TGG ATC TCA AAG AC-3′;
TNFα-R:
(SEQ ID NO: 29)
5′-AGA TAG CAA ATC GGC TGA CG-3′;
Adrp30-F:
(SEQ ID NO: 30)
5′-GCA CTG GCA AGT TCT ACT GCA A-3′;
Adrp30-R:
(SEQ ID NO: 31)
5′-GTA GGT GAA GAG AAC GGC CTT GT-3′;
C/ebprβ-F:
(SEQ ID NO: 32)
5′-ACG ACT TCC TCT CCG ACC TCT-3′;
C/ebprβ-R:
(SEQ ID NO: 33)
5′-CGA GGC TCA CGT AAC CGT AGT-3′;
PPARγ-F:
(SEQ ID NO: 34)
5′-TGA AAG AAG CGG TGA ACC ACT G-3′;
PPARγ-R:
(SEQ ID NO: 35)
5′-TGG CAT CTC TGT GTC AAC CAT G-3′;
UCP1-F:
(SEQ ID NO: 36)
5′-ACT GCC ACA CCT CCA GTC ATT-3′;
UCP1-R:
(SEQ ID NO: 37)
5′-CTT TGC CTC ACT CAG GAT TGG-3′;
Tgfb1-F:
(SEQ ID NO: 38)
5′-GGA GAG CCC TGG ATA CCA AC-3′;
Tgfb1-R:
(SEQ ID NO: 39)
5′-AAG TTG GCA TGG TAG CCC TT-3′;
αSMA-F:
(SEQ ID NO: 40)
5′-GTT CAG TGG TGC CTC TGT CA-3′;
αSMA-R:
(SEQ ID NO: 41)
5′-ACT GGG ACG ACA TGG AAA AG-3′;
Casp2-F:
(SEQ ID NO: 42)
5′-CAC CCT CTT CAA GCT TTT GG-3′;
Casp2-R:
(SEQ ID NO: 43)
5′-CGA AAA ACC TCT TGG AGC TG-3′;
Cyclophilin A-F:
(SEQ ID NO: 44)
5′-TGG AGA GCA CCA AGA CAG ACA-3′;
Cyclophilin A-R:
(SEQ ID NO: 45)
5′-TGC CGG AGT CGA CAA TGA T-3′.

Primer sequences for Tnf, Arg1, F4/80, Cd11b, Mcp1, Mgl1, Cd68 were described (Lumeng et al., 2007) and lipogenic enzyme mRNAs including mSrebf1c, Srebf2, Fasn, Scd-1, Hmgcr, Hmgcs, and Ldlr were also described (Yang et al., 2001).

Immunohistochemistry—

Liver and epidydimal fat were fixed in 4% paraformaldehyde for 48 hr and embedded in paraffin, sectioned, stained with hematoxylin and eosin (H&E) to evaluate gross morphology and ballooning degeneration and with Sirius Red to determine fibrosis. Prepared FFPE sections were subjected to incubation with antibodies specific to p62 and F4/80 to visualize formation of Mallory-Denk Bodies (MDB) and macrophage infiltration, respectively. For frozen-block preparation, liver tissue was embedded in Tissue-Tek OCT compound (Sakura Finetek), sectioned, and stained with Oil Red O to visualize TG accumulation. Stained areas were quantified using ImageJ software.

Expression Vectors—

Mouse SP (MC204593) and mouse Casp2 cDNA clone (MC206011) were purchased from Origene Technologies, Inc. Flag-tagged SREBP2 (#32018) cDNA clone was from Addgene. Myc-S1P in which the c-Myc epitope was inserted between AA23 and 24 of mouse S1P were generated as previously described (Sakai et al., 1998). S1P-Myc with a C-terminal Myc epitope was generated by PCR amplification and subsequently cloned between the Nhe1 and BamH1 sites of pCDH-CMV-MCS-EF1-Puro (#CD500-CD700, System Bioscience). Casp2-HA with a C terminal HA epitope was amplified with the following primers and subsequently cloned between the NheI and BamHI sites of pCDH-CMV-MCS-EF1-Puro.

Nhe1-Kozak-Casp2-F:
(SEQ ID NO: 46)
5′-ACACGCTAGCACCATGGCAATGGCGGCGCCGAGCGGGAGGTC-3′;
Casp2-HA-stop-BamH1-R:
(SEQ ID NO: 47)
5′-ACACGGATCCTCAAGCGTAATCTGGAACATCGTATGGGTACGTGGGT
GGGTAGCCT-3′.

Myc-S1P^{V214A/D218E}, Myc-S1P^{V721A/D725E}, Myc-S1P^{V735A/D739E}, Myc-S1P^{V842A/D846E}, and catalytically inactive Casp2-HA (Casp2^{C320G}-HA) were generated using PrimeSTAR Mutagenesis Basal kit (TaKaRa, R046A) according to the manufacturer's instructions. The sequences of each primer set were as follows:

Myc-S1P^{V214A/D218E}-F:
(SEQ ID NO: 48)
5′-GCTGCTGTTTTTGAAACTGGGCTCAGTGAGAAG-3′;
Myc-S1P^{V214A/D218E}-R:
(SEQ ID NO: 49)
5′-TTCAAAAACAGCAGCTCTGACATTAGCACCTGT-3′;
Myc-S1P^{V721A/D725E}-F:
(SEQ ID NO: 50)
5′-GCCATCTTCAGTGAGTGGTACAACACTTCTGTT-3′;
Myc-S1P^{V721A/D725E}-R:
(SEQ ID NO: 51)
5′-CTCACTGAAGATGGCGAGGGAAAGGCCATTGTC-3′;
Myc-S1P^{V735A/D739E}-F:
(SEQ ID NO: 52)
5′-GCGAAGTTTTATGAGGAAAACACCAGGCAGTGG-3′;
Myc-S1P^{V735A/D739E}-R:
(SEQ ID NO: 53)
5′-CTCATAAAACTTCGCTTTTCTCATAACAGAAGT-3′;
Myc-S1P^{V842A/D846E}-F:
(SEQ ID NO: 54)
5′-GCGCTGTATGGAGAGTCCAACTGCTTGGATGAC-3′;
Mc-S1P^{V842A/D846E}-R:
(SEQ ID NO: 55)
5′-CTCTCCATACAGCgCGATCCGGCCTCCACCTTC-3′;
Casp2^{C320G}-F:
(SEQ ID NO: 56)
5′-CAAGCAGGTCGTGGAGATGAGACAGATAGAGGT-3′;
Casp2^{C320G}-R:
(SEQ ID NO: 57)
5′-TCCACGACCTGCTTGGATGAAGAACATTTTTGG-3′.

The DNA sequences of the cDNA constructs were confirmed by DNA sequencing.

Immunofluorescence—

Cells were plated on cover slips on day 0 at density of 1.5×10⁵ cells per well. At day 1, 0.2 μg of plasmid DNA were transfected using Lipofectamine 3000. After incubation in 1% LPDS, the cells were fixed with 4% paraformaldehyde for 30 min followed by permeabilization with 0.2% Triton X-100 for 3 min. The cells were then incubated in blocking solution (5% Bovine Serum Albumin, 5% Donkey serum, 0.1% Tween 20 in PBS, pH 7.4) for 1 hr at RT, followed by O.N. incubation with primary antibodies. On the next day, the cells were washed with PBS 5 times and incubated with secondary conjugated antibodies for 1 hr. DAPI was used for nuclear staining. Anti-rat HA (1:100) and Alexa-594 conjugated donkey anti-rat IgG (A21209, Life Technologies) were used for detection of Casp2-HA, anti-mouse Myc (1:100) and Alexa-488 conjugated donkey anti-mouse IgG (A21202, Life Technologies) were used for S1P-Myc, anti-rabbit Flag (1:100) and Alexa-647 conjugated donkey anti-rabbit IgG (A31573, Life Technologies) were used to detect Flag-SREBP2. Images were taken via a Leica TCS SP5× confocal microscope.

Human Specimens—

Patients with clinical indication for liver biopsy were prospectively enrolled (Caussy et al., 2017; Loomba et al., 2017; Park et al., 2017). After undergoing evaluation for other causes of hepatic steatosis and liver disease, patients were invited to undergo standardized history, physical and anthropometric exam, and laboratory testing. Participants were included if they met the following criteria: (1) 18 years or older, (2) Fat accumulation in the liver (steatosis) involving at least 5% of hepatocytes on routine stains, (3) No evidence of other acute or chronic liver disease, and (4) Absence of regular or excessive use of alcohol. Regular or excessive alcohol is defined as an average alcohol intake of more than 14 drinks of alcohol/week in men or more than 7 drinks of alcohol/week in women. Liver histology assessment was done using the NASH CRN Histologic Scoring System by an experienced, blinded GI pathologist. All biopsies were assessed for the following three parameters: steatosis was graded 0-3, lobular inflammation was graded 0-3, ballooning was graded 0-2, fibrosis stage was classified into five staged from 0-4. Presence of NASH was defined as a pattern that was consistent with steatohepatitis including presence of steatosis, lobular inflammation and ballooning with or without perisinusoidal fibrosis. NAFL was defined as the presence of steatosis with no evidence of hepatocellular injury in the form of hepatocyte ballooning or no evidence of fibrosis. For non-NAFLD control group, non-NAFLD controls were derived from the Twin and Family cohort, prospectively recruited (Caussy et al., 2017; Loomba et al., 2017; Park et al., 2017). All participants underwent a standardized exhaustive clinical research visit including detailed medical history, physical examination, and testing to rule out other causes of chronic liver diseases (see inclusion and exclusion criteria for further details), fasting laboratory tests and then underwent an advanced Magnetic Resonance examination including magnetic resonance imaging proton density fat fraction (MRI-PDFF) for the quantification of liver fat content and for the screening of NAFLD and advanced fibrosis (Le et al., 2012; Loomba et al., 2015). Participants without evidence of NAFLD (MRI-PDFF<5%) were considered as non-NAFLD control.

Triglyceride and Cholesterol Analysis—

Liver lipids were extracted using chloroform/methanol (2:1 v/v) and plasma and liver TG and cholesterol were determined using Triglyceride Colorimetric Assay Kit (Cayman Chemical Company, MI) and Cholesterol Quantification Kit (Sigma-Aldrich, MO) according to manufacturer's instructions.

Metabolic Cage Analysis—

Indicated mice were fed with HFD for 12 weeks and subjected to metabolic cage analyses. Metabolic parameters including O₂ consumption, CO₂ production, and respiratory exchange ratio were recorded by Comprehensive Lab Animal Monitoring System (Columbus Instruments) for 4 consecutive days and nights, with at least 24 hr adaptation period prior to data recording.

ER and Golgi Isolation and Separation—

The ER and Golgi fractions were isolated from mouse liver as described previously (Croze and Morre, 1984). Briefly, mice were sacrificed and livers were quickly removed. 0.8 g of liver tissue were minced and placed in 3.0 ml of homogenization buffer (37.5 mM TRIS-maleate, pH 6.4; 0.5 M sucrose; 1% dextran; 5 mM MgCl₂). After homogenization with a Herdolph RZR 50 motordriven homogenizer, liver homogenates were centrifuged at 5,000 g for 15 min (Sorvall RC6+, SS-34 rotor). To isolate the Golgi compartment, the yellow-brown portion (upper one-third) of the pellet was removed and suspended in 0.5 ml of homogenization buffer and then layered over a 1.2 M sucrose cushion. After centrifugation at 100,000 g for 30 min (Beckman Coulter Optima XE-90, SW 55 Ti rotor), the Golgi fraction was collected from the homogenate-1.2M sucrose interface. Collected Golgi compartments were diluted in homogenization buffer and centrifuged at 5,500 g for 20 min. To isolate the ER fraction, the supernatant from the initial centrifugation was combined with the one obtained from the 100,000 g spin used to isolate the Golgi complex and subjected to manual-homogenization in 2 ml of homogenization buffer. Homogenates were centrifuged at 8,500 g for 5 min (Sorvall RC6+, SS-34 rotor) to remove mitochondria. The supernatant was layered onto a discontinuous sucrose gradient consisting of 2.0 M, 1.5 M and 1.3 M sucrose in a v/v ratio of 3:4:4 and centrifuged for 120 min at 90,000 g (Beckman Coulter Optima XE-90, SW 41 Ti rotor). ER fraction was collected from the 1.3M-1.5M interface and the 1.5M-2.0M interface. Proteins in each of the membrane fractions were extracted by sonication, de-glycosylated, and subjected to IB analysis as described.

In Vitro Adipocyte Differentiation—

Preadipocytes were obtained from inguinal adipose tissue, and induced to differentiate in DMEM supplemented with 10% FBS along with 500 μM 3-Isobutyl-1-methylxanthine, 2.5 μM dexamethasone, 1 μg/ml insulin and 504 Rosiglitazone. After 3 days, the cells were cultured in DMEM supplemented with 10% FBS and 1 μg/ml insulin for another 3 days. Then, mature adipocytes were kept in 10% FBS containing DMEM.

De Novo Lipogenesis—

Two days after completion of the differentiation process, adipocytes were starved overnight and then incubated for 2 hr at 37° C. in DMEM-0.2% fatty acid-free BSA with or without 50 nM insulin and 0.5 μCi ¹⁴C-glucose. After incubation, the cells were washed with PBS for 3 times, and lysed in 200 μl of 0.1 N HCl. Lipids were extracted by adding 500 μl of 2:1 chloroform-methanol to 100 μl cell lysate. After 5 min incubation, 250 μl water was added. Samples were centrifuged at 3,000 g for 10 min. 100 μl lower phase was transferred into 5 ml liquid scintillation fluid to measure ¹⁴C activity. ¹⁴C activity was normalized to cellular protein content

Morphological Analysis of Fat—

Morphological analysis of adipocytes was performed by using MRI adipocyte tools (Osman et al., 2013). Images were taken from FFPE epidydimal fat section by AXIO Imager A2 (Carl Zeiss, Germany) and subjected to MRI adipocyte software to obtain area of individual fat cell and number of adipocytes per HMF. Quantification and statistical significance were analyzed by Prism 7 software (GraphPad Prism, CA).

### Example 2

### Caspase 2 Expression is Induced by ER Stress

ER stress in MUP-uPA liver peaks in 5- to 6-week-old mice and then declines due to reduced uPA expression in newly regenerated hepatocytes (Nakagawa et al., 2014; Sandgren et al., 1991). The livers of young MUP-uPA mice were examined for expression of different caspases, including Casp2, Casp3, Casp4, Casp8, and Casp12, and it was found that particularly high amounts of precursor and cleaved forms of Casp2 (FIGS. 12A and 12G), as well as more Casp2 mRNA relative to non-transgenic controls (FIG. 12B). Older NC-fed MUP-uPA mice, in which liver ER stress is low, showed elevated Casp2 mRNA after HFD feeding (FIG. 12C). This response was attenuated in TNF receptor 1 (TNFR1)-deficient mice, which are protected from HFD-induced NASH development (Nakagawa et al., 2014). HFD feeding also increased Casp2 protein expression, but to a lesser extent than its effect on mRNA expression (FIG. 12G). Administration of the ER stress inducer tunicamycin to non-transgenic 3-month-old NC-fed BL6 mice also induced Casp2 protein and RNA (FIGS. 12A and 12D). Elevated Casp2 expression in NC-fed young and HFD-fed adult MUP-uPA mice was also detected by immunohistochemistry (IHC) and confirmed by PCR analysis, showing that HFD feeding also induced Tnf mRNA (FIGS. 12H and 121).

Human liver biopsies were also examined for CASP2 expression. Notably, CASP2 expression was highly elevated in NASH biopsies relative to those from simple steatosis (FIG. 12E). CASP2 staining of human or mouse NASH was not associated with any apoptotic or degenerative features other than macrovesicular fat and hepatocyte ballooning. RNA from liver biopsies taken from simple steatosis and NASH patients was analyzed and it was confirmed that CASP2 mRNA was higher in NASH, which also exhibited elevated expression of several ER stress markers, including HSPA5 (GRP78/BIP) and ATF6 (FIG. 12F). These results support the suitability of the mouse model described herein for studying how ER stress drives NASH pathogenesis.

### Example 3

### Caspase 2 is Required for NASH Development

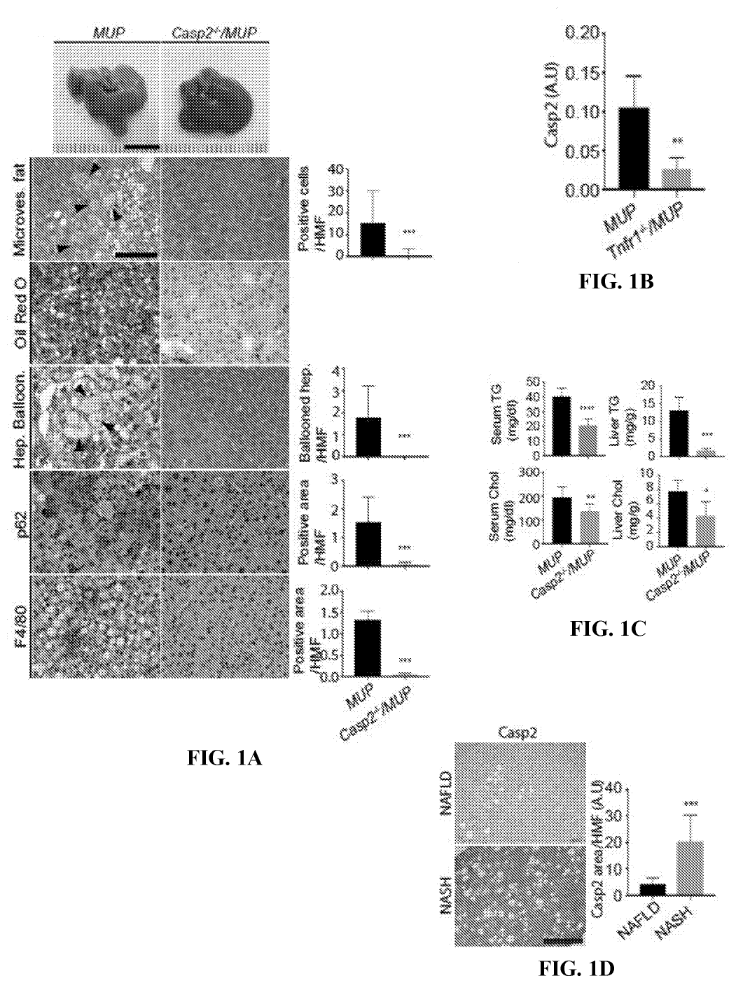

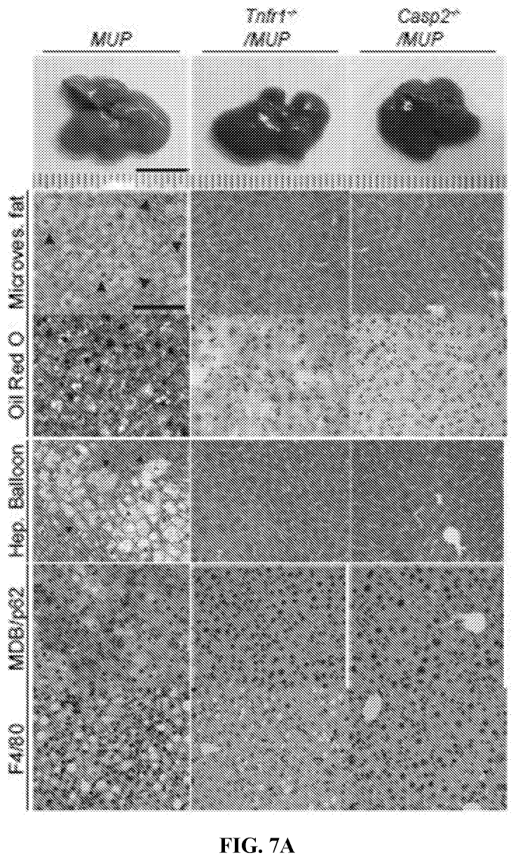

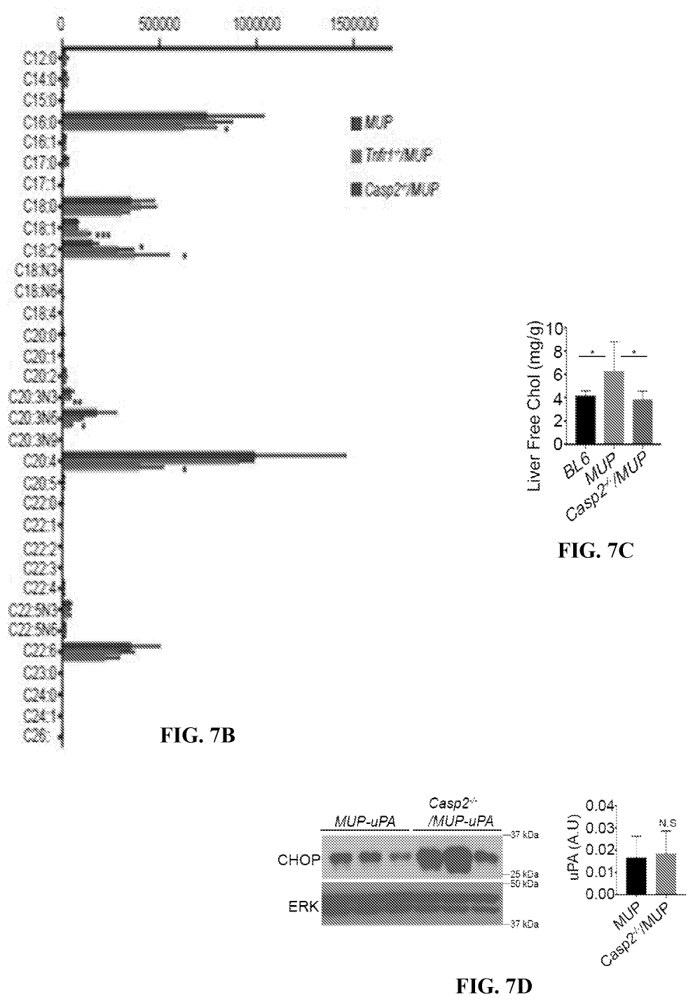

Casp2-deficient MUP-uPA mice (Casp2^{-/-}/MUP-uPA), which were born at the expected Mendelian ratio without obvious growth retardation or illness were generated. To investigate the role of Casp2 in NASH progression, 8 week old MUP-uPA and Casp2^{-/-}/MUP-uPA mice were placed on HFD, in which 60% of the caloric value is provided by fats (mostly saturated). After 12 weeks of HFD-feeding, livers were excised and NASH signs were evaluated. Casp2^{-/-}/MUP-uPA mice did not exhibit fatty liver and were protected from lipid droplet accumulation, ballooning degeneration, Mallory-Denk body (MDB) formation, p62 accumulation and macrophage infiltration (FIG. 1A). Previously, it was found that TNF type 1 receptor (TNFR1) ablation in MUP-uPA mice (Tnfr1^{-/-}/MUP-uPA) also attenuated NASH progression (Nakagawa et al., 2014). The protective effect of Casp2 ablation was as pronounced as that of TNFR1 ablation (FIG. 7A), which reduced Casp2 mRNA expression by 70% (FIG. 1B). Casp2 ablation reduced serum and liver triglycerides (TG) by 50% and 85%, respectively (FIG. 1C). Serum and liver cholesterol were reduced by ˜20% and 50%, respectively. Analysis of serum TG profiles indicates that C16:0 was reduced, but linoleic acids including C18:1 and C18:2 were increased in Casp2-ablated MUP-uPA mice (FIG. 7B). An even bigger reduction was seen in the serum content of C20:3n3, C20:3n6, and C20:4 in Casp2^{-/-}/MUP-uPA mice.

To explore the relevance of Casp2 to human NASH, NAFLD and NASH liver, biopsies were immunostained. Notably, Casp2 expression was highly elevated in NASH relative to simple steatosis (FIG. 1D). Moreover, elevated Casp2 staining was not associated with any apoptotic or degenerative features other than macrovesicular fat and hepatocyte ballooning. Casp2 ablation also brought down liver free (non-esterified) cholesterol in HFD-fed MUP-uPA mice to the same level as in BL6 mice (FIG. 7C). In 5-week-old MUP-uPA mice, which express high amounts of uPA and therefore undergo ER stress, Casp2 ablation had no effect on uPA mRNA expression and did not block activation of the ER stress/UPR response, which based on CHOP protein expression was elevated in Casp2^{-/-}/MUP-uPA mice (FIG. 7D).

### Example 4

### Casp2 Regulates Adipose Tissue Expansion

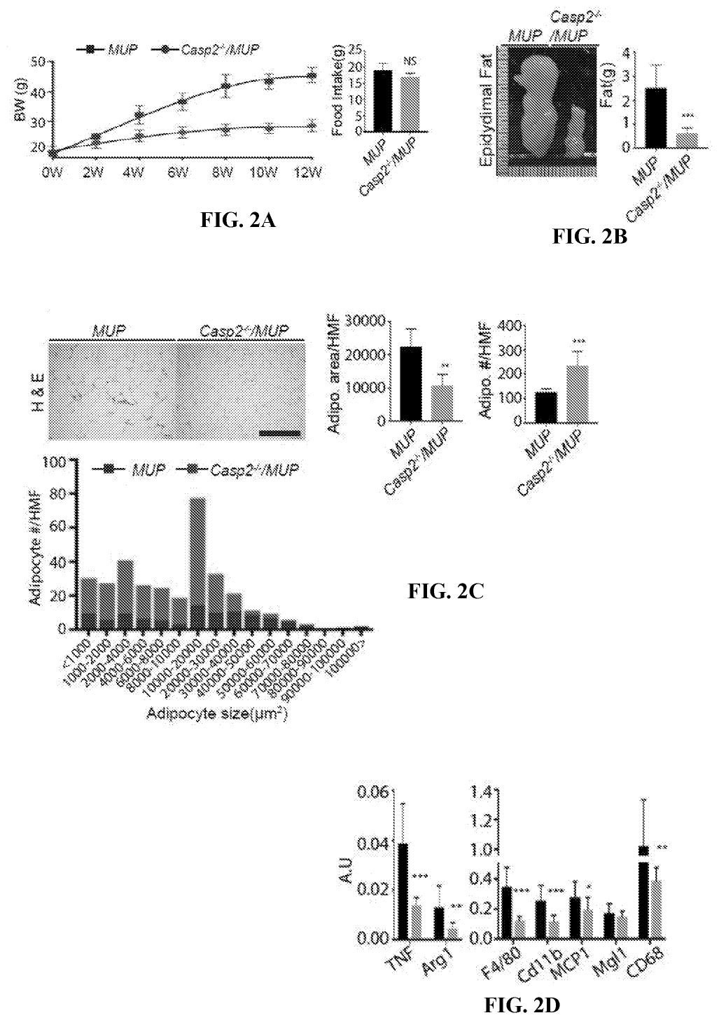

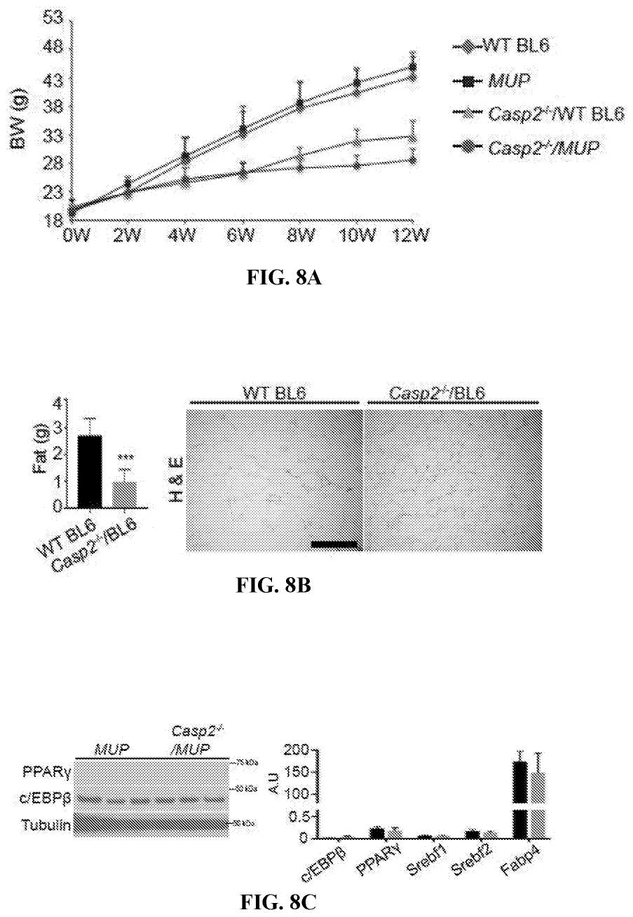

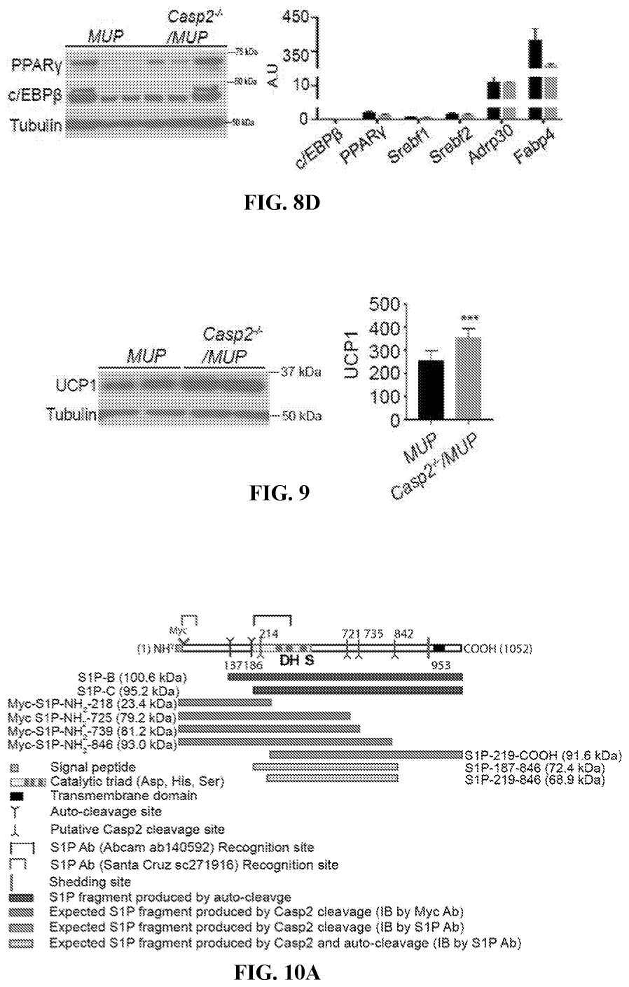

Mouse body weight and food consumption was recorded every two weeks throughout the HFD-feeding period. MUP-uPA mice gained weight gradually and did not differ from nontransgenic WT BL6 mice (FIG. 8A). Remarkably, Casp2 ablation in either MUP-uPA or BL6 mice inhibited weight gain without any effect on food consumption (FIG. 2A; FIG. 8A). Casp2 ablation also prevented fat depot expansion (FIG. 2B) in both MUP-uPA and BL6 mice (FIG. 8B). Morphological analysis indicated that epidydimal adipocytes in HFD-fed MUP-uPA mice were twice as large as Casp2^{-/-}/MUP-uPA adipocytes (FIG. 2C). Increased adipogenesis and enlarged adipocytes are associated with adipose tissue inflammation (Xu et al., 2003). Indeed, expression of macrophage markers and inflammatory cytokine mRNAs was reduced in Casp2^{-/-}/MUP-uPA mice (FIG. 2D).

Adipogenesis is controlled by an elaborate network of transcription factors that coordinate expression of genes whose products convert preadipocytes to mature fat cells, including PPARγ and c/EBP family members (Rosen et al., 2000). To investigate the effect of Casp2 on adipogenesis, stromal vascular fractions (SVF) that contain adipocyte precursors were isolated and differentiated into adipocytes. Expression of PPARγ and c/EBPβ as well as key lipogenic factors was examined. Surprisingly, PPARγ and C/EBPβ expression in Casp2-deficient adipocytes was similar to that in MUP-uPA adipocytes and expression of the adipogenic marker transcripts Srebf1a, Srebf2, Adrp30, and Fabp4 was not altered either (FIGS. 8C and 8D). It was therefore concluded that Casp2 controls adipose tissue expansion by regulating hypertrophy rather than adipogenesis per se, having no effect on PPARγ and C/EBPβ.

### Example 5

### Casp2 Ablation Increases Energy Expenditure

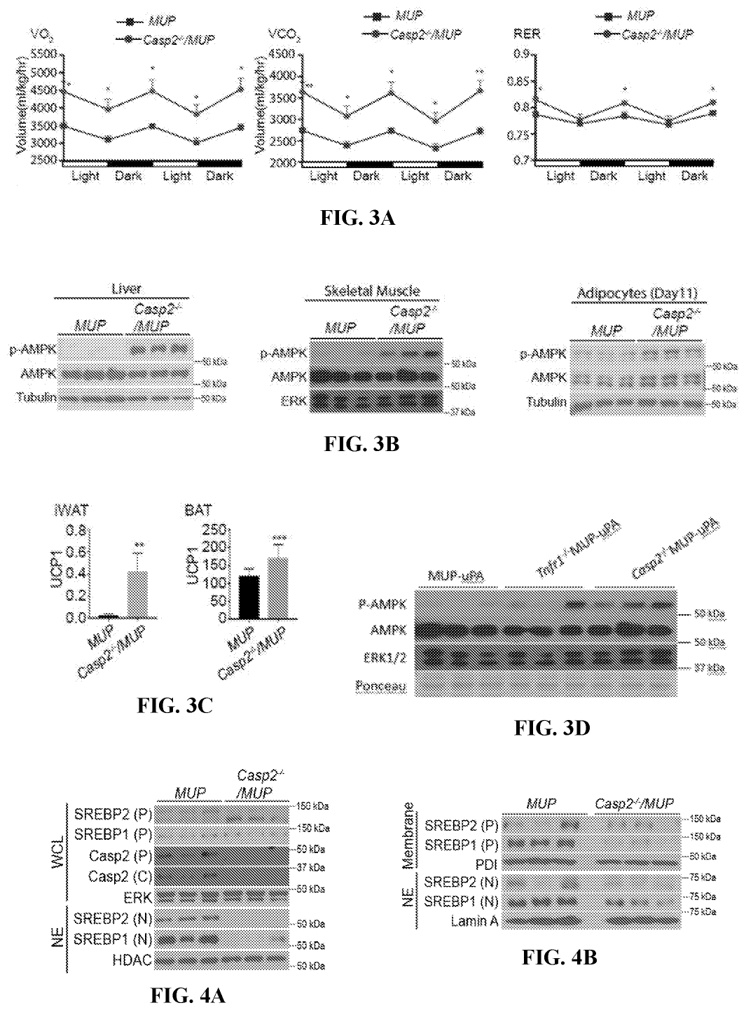

Since Casp2^{-/-}/MUP-uPA mice consumed as much food as MUP-uPA mice, it was postulated that the failure to gain weight and fat could be due, at least in part, to increased energy expenditure. Indeed, Casp2 ablation dramatically increased oxygen consumption, CO₂ production, with a modest increase in respiratory exchange rate (RER), indicating elevated energy expenditure (FIG. 3A). Congruently, Casp2-deficient mice exhibited elevated AMPK phosphorylation in metabolically active tissues, including liver, skeletal muscle and fat (FIGS. 3B and 3D). UCP1-mediated thermogenesis protects humans and mice from obesity and obesity-associated diseases (Feldmann et al., 2009; Inamine et al., 2016). Moreover, adipocyte-specific FA synthase (FAS) ablation enhances UCP1-mediated thermogenesis (Lodhi et al., 2012). Consistent with SREBP1 inhibition and reduced DNL (see below), Casp2 ablation increased UCP1 expression in inguinal fat and brown adipose tissue of MUP-uPA mice (FIG. 3C). In addition, brown adipose tissue whitening induced by HFD was reversed in Casp2^{-/-}/MUP-uPA mice. HFD-fed MUP-uPA and Casp2^{-/-}/MUP-uPA mice were also subjected to acute cold exposure and it was found that Casp2^{-/-}/MUP-uPA mice expressed more UCP1 in brown adipose tissue (FIG. 9).

### Example 6

### Casp2 Controls SREBP Cleavage and Lipogenic Gene Expression

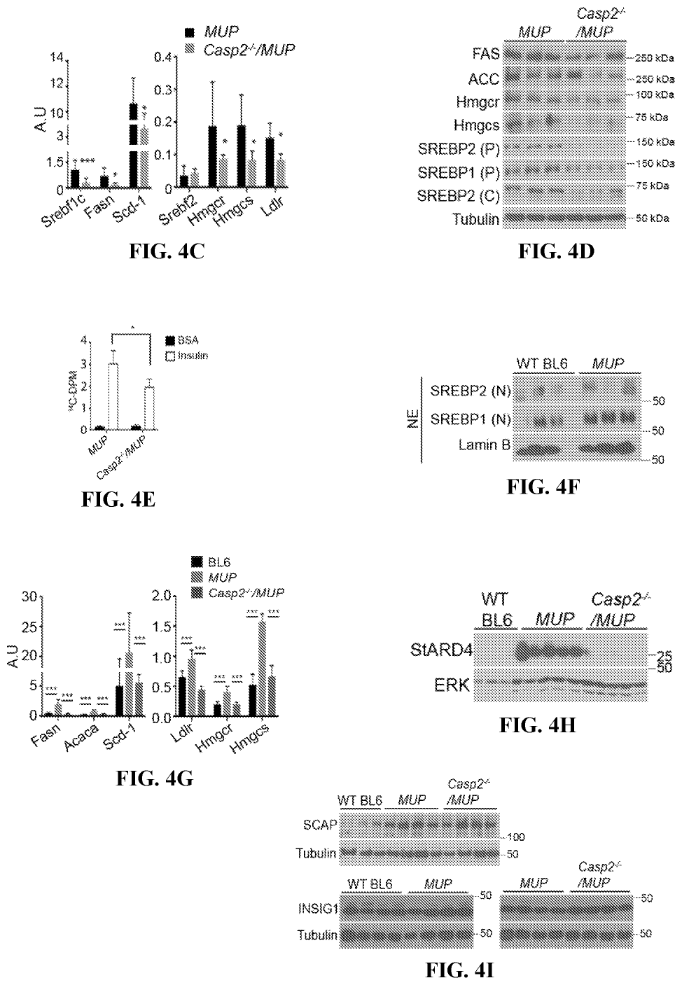

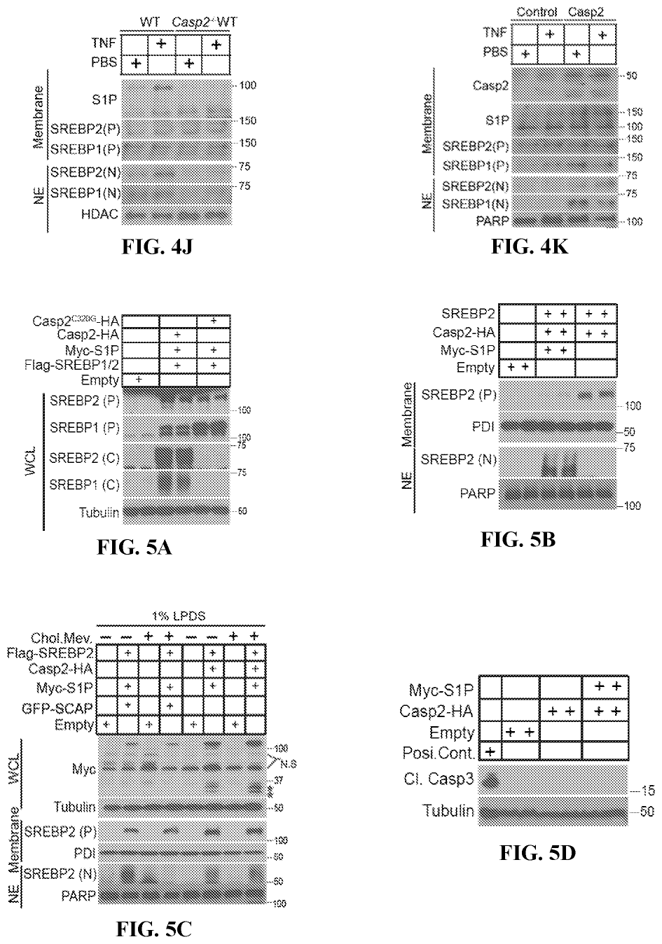

To test the effect of Casp2 on SREBP-dependent lipogenesis, SREBP1/2 activation was examined in 5 week old MUP-uPA mice. As found previously (Nakagawa et al., 2014), young MUP-uPA mice contain nuclear SREBP1 and SREBP2 in hepatocytes along with the precursor and cleaved forms of Casp2 (FIG. 4A).

The livers from MUP-uPA mice and Casp2^{-/-}/MUP-uPA mice were collected at 5 weeks of age, a time point at which MUP-uPA livers are inflamed, steatotic and Casp2 is activated, as it is in human NASH. To prepare whole cell lysates (WCL), 20 mg of liver tissue cut into small pieces was homogenized in a lysis buffer that contains 1% of Triton X-100. Homogenates were centrifuged to get rid of unbroken cells. The supernatants were saved, and protein amounts were evaluated by BCA analysis. Nuclear fraction was obtained from homogenates of 40 mg tissue. Nuclear fractions were saved from another round of centrifugation. Protein amounts were determined by BCA analysis. 40 ug of prepared WCL or nuclear extract were separated by SDS-PAGE and subjected to IB analysis to detect SREBP and Casp2. It was found that Casp2-ablation completely suppressed the appearance of active N-terminal fragments of SREBP in the nucleus (FIG. 12A), suggesting that Casp2 is required for SREBP cleavage in liver. Additionally, it was found that Casp2 ablation abolished SREBP1/2 processing and activation (of note, the antibodies used do not discriminate between SREBP1a and SREBP1c) in young MUP-uPA mice and also reduced nuclear SREBP1/2 in adult MUP-uPA mice fed with HFD for 12 weeks (FIGS. 4A and 4B). TNFR1 ablation also inhibited SREBP1 activation and attenuated activation of SREBP2 (FIG. 4B).

Generally, nuclear SREBP1 and SEREBP2 were higher after HFD feeding in MUP-uPA mice than in HFD-fed BL6 mice (FIG. 4F). Consistent with inhibition of SREBP1/2 processing, expression of several SREBP target genes, including Srebf1c itself, Fasn, Scd-1, Hmgcr, Hmgcs, and Ldlr were significantly lower in Casp2^{-/-}/MUP-uPA livers than in MUP-uPA livers (FIG. 4C). In fact, Casp2 ablation reduced expression of these genes to the same level seen in HFD-fed BL6 mice (FIG. 4G), suggesting it reversed the effect of uPA-induced ER stress. Casp2 ablation also resulted in a marked decrease in expression of StARD4, a regulator of intracellular cholesterol transport, which was highly elevated in MUP-uPA mice (FIG. 4H). Casp2 ablation, however, had no effect on expression of either INSIG1 or SCAP in HFD-fed MUP-uPA mice, which expressed as much INSIG1 as WT mice (FIG. 4I). Casp2 expression was also needed for TNF-induced SREBP1 and 2 activation in primary WT hepatocytes (FIG. 4J). Re-expression of Casp2 in Casp2-null hepatocytes restored SREBP1 and 2 activation, whether or not the cells were incubated with TNF (FIG. 4K).

To determine the effect of Casp2 on SREBP1/2 and lipogenesis in adipocytes, preadipocytes in SVF collected from MUP-uPA and Casp2^{-/-}/MUP-uPA mice were differentiated into mature adipocytes. The absence of Casp2 reduced expression of precursor and cleaved forms of SREBP1/2, as well as lipogenic enzymes (FIG. 4D) and decreased insulin-induced adipocyte lipogenesis (FIG. 4E). These results demonstrate that Casp2 regulates SREBP1/2 activation and DNL not only in hepatocytes but also in adipocytes.

### Example 7

### Casp2 Cleaves SW but not SREBPs

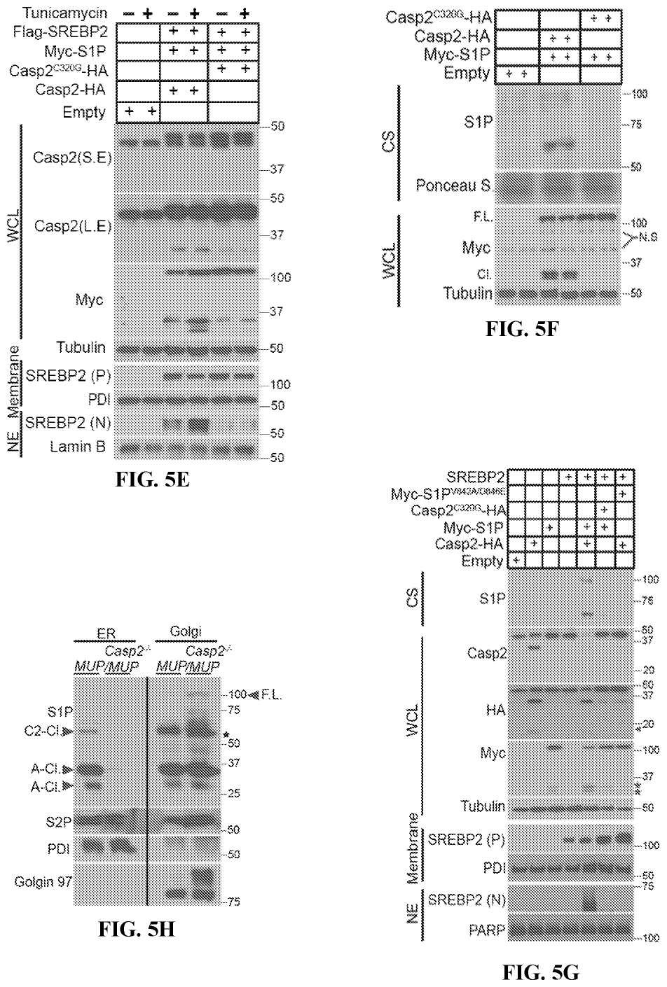

Apoptopic caspases including Casp3 and Drice directly cleave and activate mammalian and fly SREBPs (Amarneh et al., 2009; Wang et al., 1996). Nonetheless, the SREBP1 and 2 primary sequences are devoid of obvious Casp2 cleavage sites, whose consensus is VDVAD (SEQ ID NO: 1) (Talanian et al., 1997). Moreover, co-transfection of Casp2 with SREBP2 into either SCAP-deficient (293^{ΔSCAP}) or WT HEK293 cells did not result in SREBP1/2 activation/cleavage, unless an SP expression vector was also included (FIGS. 5A and 5B). Notably, SREBP activation under these conditions was dependent on Casp2 catalytic activity, but independent of SCAP, as it was almost as pronounced in 293^{ΔSCAP} cells as in WT HEK293 cells. In WT HEK293 cells incubated in lipoprotein-deficient serum, SREBP2 was activated by co-transfection of SP with SCAP, and the response was strongly inhibited by incubation with cholesterol plus mevalonate. By contrast, activation of SREBP2 by Casp2 plus SP was completely refractory to feedback inhibition. Importantly, co-expression of Casp2 with S1P or SREBP2 in S1P-deficient HEK293 cells resulted in colocalization of Casp2 with SP and SREBP2 in juxta-nuclear structures probably corresponding to peri-nuclear ER and early Golgi. However, expression of Casp2 alone resulted in diffuse cytoplasmic staining and under no circumstance did Casp2 expression, without or with S1P, result in apoptotic morphology or Casp3 activation (FIG. 5D). ER stress was reported to induce SREBP activation (Colgan et al., 2011). Casp2 activity was needed for SREBP2 activation by tunicamycin, a potent ER stress inducer, in S1P-transfected HEK293 cells (FIG. 5E).

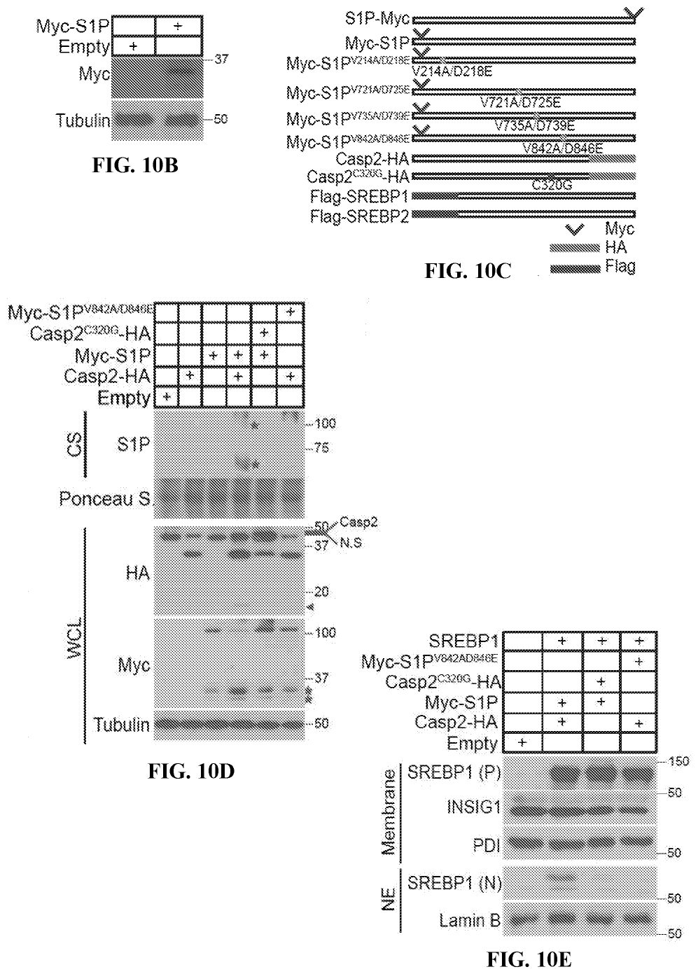

Under most conditions, SREBP1/2 processing depends on S1P and S2P (Brown and Goldstein, 1997b). The SW precursor is an ER membrane-anchored 1052 amino acid protein that contains two autocleavage sites: ¹³⁴RSLK¹³⁷ (SEQ ID NO: 2) and ¹⁸³RRLL¹⁸⁶ (SEQ ID NO: 3). Cleavage at these sites generates B- and C-form S1P, which remain membrane-anchored and whose sizes are 100 and 95 kDa, respectively (FIG. 10A). Active C-form S1P has been shown to translocate to the Golgi apparatus, from which it can be secreted to the extracellular millieu (Cheng et al., 1999; Espenshade et al., 1999).

Coexpression of catalytically active Casp2 with S1P in WT HEK293 generated a new 68 kDa S1P polypeptide that was secreted into the culture supernatant (FIG. 5F), akin to autocleaved C-form SP whose C-terminal transmembrane (TM) domain was removed by mutagenesis (Cheng et al., 1999). Curiously, coexpression of Casp2 with SP also led to appearance of a larger secreted polypeptide migrating around 100 kDa, suggesting that this S1P form and the 68 kDa form lack the C-terminal TM domain (FIGS. 5F and 5G). Casp2 also enhanced formation of 2-3 smaller intracellular S1P polypeptides migrating ca. 30 kDa that, unlike the secreted 68 and 100 kDa polypeptides, retained the N-terminal Myc epitope that was inserted after the signal sequence (FIGS. 5C, 5F and 10A). These polypeptides are likely generated via S1P autocleavage, because they were also present in S1P-overexpressing HEK293 cells without Casp2 coexpression (FIG. 10B).

To better understand the origin of these S1P forms, microsomal fractions that contain ER membranes in which S1P auto-proteolysis is initiated were isolated from livers of MUP-uPA, Casp2^{-/-}/MUP-uPA, and Tnfr1^{-/-}/MUP-uPA mice. Of note, abundance of the 100 kDa form of S1P was lower in membranes from Casp2 deficient mice (FIG. 11A). Incubation of isolated MUP-uPA hepatocytes with TNF induced appearance of 100 kDa S1P in membrane fractions and cleaved SREBP1/2 in the nuclear fraction. To address whether Casp2 regulates SREBP cleavage through SP activation in hepatocytes, primary hepatocytes were freshly isolated from WT mice or Casp2^{-/-}WT mice. After 24 hrs, hepatocytes were incubated in either the presence of TNF (10 ng/ml) or PBS for 16 hrs. Prepared hepatocytes were collected in a hypotonic buffer and passed through 221/2 G syringe more than 20 times to break the cells. The homogenate was subjected to sequential centrifugations to separate membrane fraction and nuclear fraction. After protein amount determination, 40 μg of protein were subjected to IB analysis to detect SREBP and S1P. It was found that TNF stimulation induces activation of S1P and cleavage of SREBP in WT hepatocytes but not in Casp2-ablated hepatocytes.

The secreted 68 kDa and 100 kDa fragments, which react with an SW antibody that recognizes an epitope located between amino acid 200 and amino acid 300 (Abcam technical information) most likely correspond to a 72 kDa polypeptide stretching from amino acid 187, generated by autocleavage at amino acid 186, to the putative Casp2 site at amino acid 842 (see below) and a longer polypeptide whose N-terminus may correspond to that of A- or B-form S1P, which is also secreted to the culture medium after TM domain removal (da Palma et al., 2014). Both forms contain the entire SP catalytic pocket, although cleavage at the A site does not generate active S1P (da Palma et al., 2014).

To better understand the relationship between Casp2 and SW processing, Myc-tagged S1P, HA-tagged Casp2, and Flag-tagged SREBP expression vectors were generated (FIG. 10C). Myc-tagged SP was co-transfected with either HA-tagged Casp2 or catalytically inactive Casp2 into HEK293T cells. A similar experiment including Flag-tagged SREBP1/2 along with the Casp2 and Myc-S1P expression vectors was also conducted. When all three proteins were co-expressed, catalytically active Casp2 induced S1P processing, leading to truncation of its secreted form and led to the cleavage of SREBP1/2 (FIG. 11B).

To determine whether Casp2-induced SP processing and SREBP activation are due to direct SP cleavage and to identify the location of the relevant cleavage site(s), the four putative Casp2 cleavage sites were altered: ²¹⁴VAVFD²¹⁸ (SEQ ID NO: 4), ⁷²¹VIFSD⁷²⁵ (SEQ ID NO: 5), ⁷³⁵VKFYD⁷³⁹ (SEQ ID NO: 6), and ⁸⁴²VLYGD⁸⁴⁶ (SEQ ID NO: 7) (FIG. 10A) by site-directed mutagenesis, replacing the Val and Asp residues at the P1 and P5 positions with Ala and Glu residues, respectively (FIG. 10C). The only mutation with a clear effect on S1P processing was the ⁸⁴²V/A-D/E⁸⁴⁶ substitution that blocked appearance of the secreted 68 and 100 kDa fragments, and reduced production of the smaller intracellular Myc tagged 30 kDa fragments (FIGS. 5G and 10D). Coexpression of WT SP with Casp2 also inhibited or reduced the expression of full-length S1P present in the whole cell lysate, but the ⁸⁴²V/A-D/E⁸⁴⁶ mutation blocked this effect (FIGS. 5G and 10D). Most importantly, the ⁸⁴²V/AD/E⁸⁴⁶ mutation prevented SREBP1 and 2 activation by Casp2 and SP (FIGS. 5G and 10E), but had no effect on SREBP activation by SCAP+S1P (FIG. 10F). These results suggest that Casp2 first cleaves S1P at amino acid 846, separating its catalytic pocket from the C-terminal TM domain, generating soluble S1P isoforms that freely diffuse throughout the ER-Golgi lumen and are capable of initiating SCAP-independent SREBP processing that is refractory to feedback inhibition. This cleavage event results in S1P activation and enhances subsequent self-cleavage at amino acid 186, which generates the secreted 68 kDa polypeptide. Small amounts of A- or B-form SP cleaved by Casp2 at amino acid 846 are also secreted to the culture medium. Cleavage at amino acid 186 may also be responsible for generating one of the short 30 kDa S1P polypeptides that contain the N-terminal Myc epitope. Of note, the small 30 kDa autocleaved forms of SP were also detected using an antibody against an N-terminal epitope in 5-week-old MUP-uPA mice, but were absent in age matched Casp2^{-/-}/MUP-uPA mice (FIG. 10G). By contrast, Casp2^{-/-}/MUP-uPA livers contained much higher amounts of membrane-associated full-length S1P, similar to what was observed in HEK293 cells. To determine the subcellular site of SP cleavage, the ER and Golgi fractions were isolated and separated from 7-week-old MUP-uPA and Casp2^{-/-}/MUP-uPA livers. Notably, Casp2 expression led to formation of cleaved SP in the ER of the MUP-uPA liver (FIG. 5H). Furthermore, only the Casp2-null MUP-uPA liver contained the membrane-bound full-length form of SP and this form was only present in the Golgi fraction. No SP polypeptides were present in the ER of the Casp2-deficient liver.

Notably, both the 68 and 100 kDa secreted S1P polypeptides were present in MUP-uPA sera, but not in Casp2^{-/-}/MUP-uPA sera (FIG. 5J). Strikingly, sera from NASH patients also contained the 68 kDa secreted S1P polypeptide, which was present in much lower amounts in sera of non-fibrotic NAFLD patients and nearly absent in normal controls (FIG. 5J). It was also examined whether S2P-mediated cleavage was required for Casp2-induced SREBP activation and generated S2P-deficient HEK293 cells. Transfection of a catalytically active Casp2 vector into these cells together with SW and SREBP2 expression vectors failed to induce SREBP2 processing, which was readily detected in WT cells (FIG. 10H). Instead, Casp2 and SW in 293^{ΔS2P} cells induced appearance of partially-processed SREBP2 migrating around 80 kDa.

### Example 8

### Effect of Casp2-Dependent S1P-SREBP Activation on NASH Progression

The effect of Casp2-dependent S1P-SREBP activation on NASH progression in HFD-fed MUP-uPA mice was then investigated. MUP-uPA, Tnfr1^{-/-}MUP-uPA and Casp2^{-/-}MUP-uPA mice were kept on HFD for up to 4 months, with the amount of food consumed and body weight being measured every 2 weeks. Mice were sacrificed after 4 months of HFD feeding and their livers and fat deposits were removed for histological and biochemical analyses. It was found that Casp2- or Tnfr1-ablation in MUP-uPA mice improved gross liver morphology (FIG. 4B) and reduced nuclear SREBP activation, especially SREBP1 which controls triglyceride synthesis. Serum cholesterol and triglyceride were reduced in Casp2-ablated mice and to a lesser extent in Tnfr1-ablated mice (FIG. 1C). Macrovesicular steatosis, macrophage infiltration, ballooning degeneration of hepatocytes and formation of Mallory-Denk bodies were significantly decreased in the absence of Casp2 or Tnfr1. Moreover, it was found that HFD-fed Casp2^{-/-}MUP-uPA mice exhibited improved glucose tolerance compared to Casp2 expressing counterparts, in part due to increased thermogenic regulation (UCPI and Pgc-1α expression) in brown adipose tissue (BAT).

### Example 9

### Casp2 Inhibitor Reverses NASH

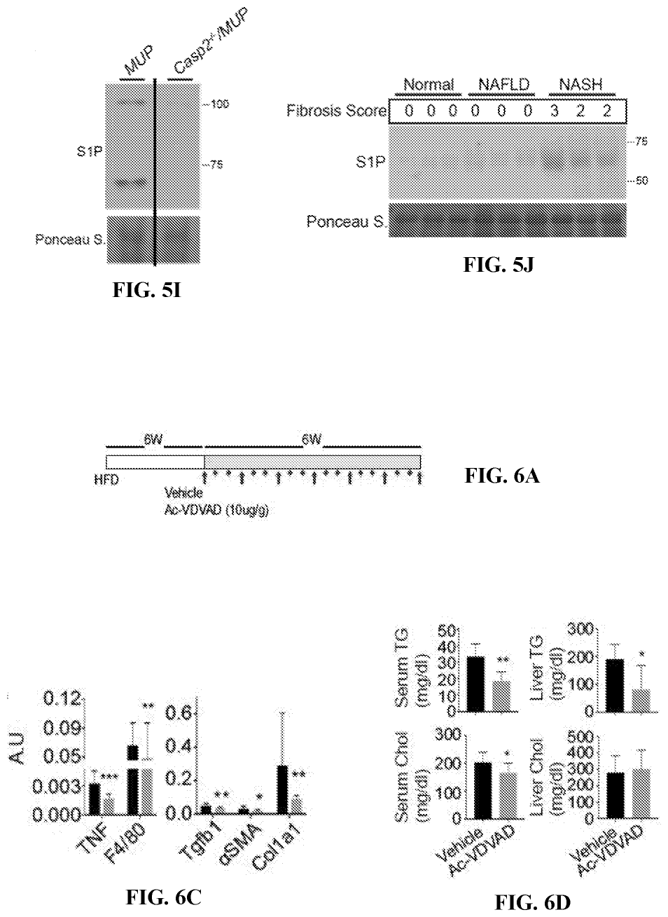

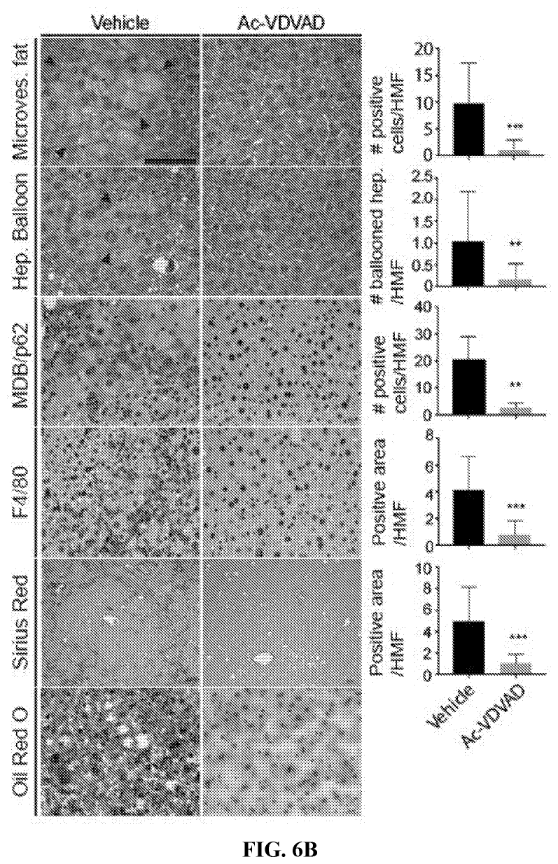

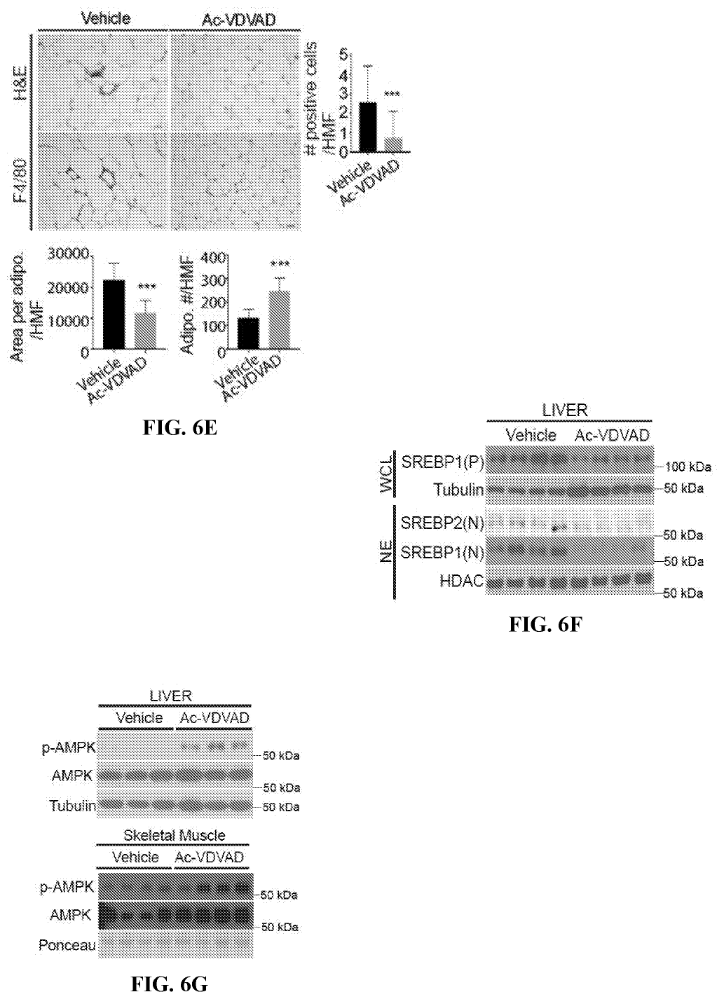

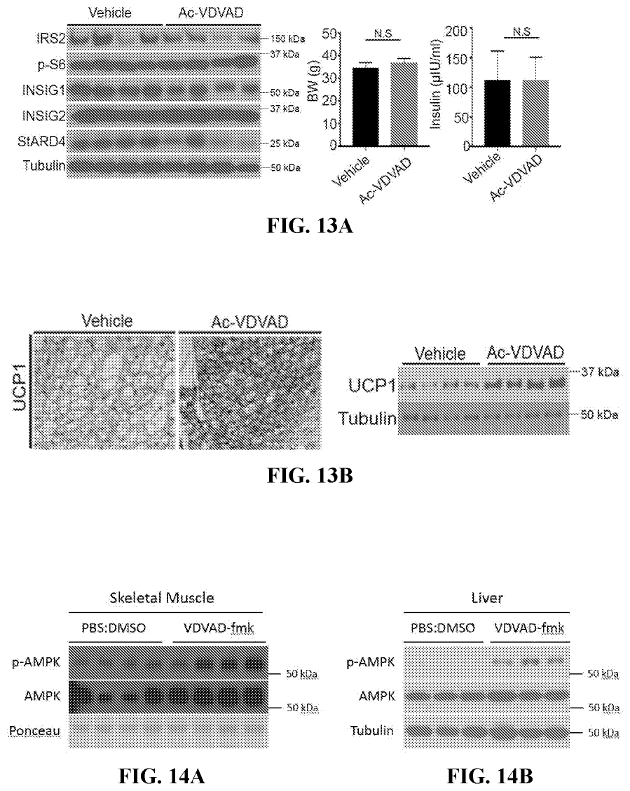

To examine whether Casp2 inhibition is of therapeutic value, Ac-VDVAD-CHO (C₂₃H₃₇N₅O₁₀), a cell permeable, Casp2 inhibitor (Talanian et al., 1997) was administrated to HFD-fed MUP-uPA mice. HFD feeding was initiated at eight weeks of age, and six weeks later the mice were given the Casp2 inhibitor on alternate days by intraperitoneal (i.p.) injection (FIG. 6A). After a 6-week treatment regimen, the mice were sacrificed and their liver and adipose tissue were examined. Treatment with the Casp2 inhibitor prevented hepatic steatosis, ballooning degeneration, p62 accumulation, and MDB formation, while decreasing macrophage recruitment (FIG. 6B). Fibrosis was also attenuated. qRT-PCR analysis confirmed that expression of inflammatory cytokines and markers as well as fibrosis-related genes was reduced in response to Casp2 inhibition (FIG. 6C). Treatment with the Casp2 inhibitor decreased circulating TG and cholesterol and liver TG (FIG. 6D) and blocked adipocyte enlargement and ATM accumulation (FIG. 6E). Consistent with these results, Casp2 inhibition attenuated SREBP1/2 activation in liver and increased energy expenditure, as suggested by AMPK activation in peripheral tissues, including liver and skeletal muscle (FIGS. 6F and 6G). Consistent with inhibition of SREBP1/2 activation, Ac-VDVAD administration reduced StARD4 but had no effect on INSIG1 or 2 expression, mTORC1/p70S6K activation, IRS2 expression, or blood insulin and body weight (FIG. 13A). The Casp2 inhibitor enhanced UCP1 expression in BAT (FIG. 13B). These results strongly suggest that Casp2 inhibition is both of a prophylactic and therapeutic value, capable of preventing NASH progression without causing hypertriglyceridemia. Furthermore, inhibition of SREBP1/2 activation is not due to any effects on insulin signaling or INSIG1/2 expression.

In a similar experiment, z-VDVAD-fmk (C₃₂H₄₆FN₅O₁₁), another cell permeable, irreversible Casp2 inhibitor, was injected into HFD-fed MUP-uPA mice. HFD feeding was started at 8 weeks of age, and six weeks later, the mice were injected intraperitoneally (i.p.) with either PBS:DMSO or z-VDVAD-fmk (10 μg/g) every other day (FIGS. 14A and 14B). After six weeks of treatment, the mice were sacrificed and their liver and adipose tissue were collected. It was found that z-VDVAD-fmk injection to HFD-fed MUP-uPA mice significantly prevented hepatic TG accumulation, decreased inflammatory infiltration and Mallory-Denk body formation in livers. Treatment with the Casp2 inhibitor dramatically reduced liver fibrosis, indicating the beneficial role of Casp2 suppression in NASH progression. Moreover, treatment with the Casp2 inhibitor decreased circulating TG and cholesterol and reduced adipose tissue macrophage (ATM) infiltration into epidydimal fat. The Casp2 inhibitor also increased thermogenesis by elevating UCP1 expression in BAT.

These results indicate that Casp2 inhibition is of a therapeutic value, being capable of reversing NASH progression and DNL without causing hypertriglyceridemia. Correlative, it was demonstrated that suppression of Casp2 activity prevents HFD-fed MUP-uPA mice from NASH progression by inhibiting hepatic lipid accumulation. Furthermore, Casp2 suppression in HFD-fed MUP-uPA mice dissipates surplus energy by increasing thermogenesis in BAT and blocking metabolic perturbations that lead to NASH progression.

The following references are incorporated herein by reference in their entireties:
Amarneh, et al. (2009). Activation of sterol regulatory element-binding protein by the caspase Drice in Drosophila larvae. J Biol Chem 284, 9674-9682.
Bobrovnikova-Marjon, et al. (2008). PERK-dependent regulation of lipogenesis during mouse mammary gland development and adipocyte differentiation. Proc Natl Acad Sci USA. 105, 16314-16319
Bouchier-Hayes, L., and Green, D. R. (2012). Caspase-2: the orphan caspase. Cell Death Differ 19, 51-57.
Brown, M. S., and Goldstein, J. L. (1997a). The SREBP pathway: regulation of cholesterol metabolism by proteolysis of a membrane-bound transcription factor. Cell 89, 331-340.
Brown, M. S., and Goldstein, J. L. (1997b). The SREBP Pathway: Regulation of Cholestrol Metabolism by Proteolysis of a Membrane-Bound Transcription Factor. Cell 89, 331-340.
Browning, J. D., and Horton, J. D. (2004). Molecular mediators of hepatic steatosis and liver injury. J Clin Invest 114, 147-152.
Caballero, et al. (2009). Enhanced free cholesterol, SREBP-2 and StAR expression in human NASH. J Hepatol 50, 789-796.
Caussy, et al. (2017). Nonalcoholic fatty liver disease with cirrhosis increases familial risk for advanced fibrosis. J Clin Invest 127, 2697-2704.
Cheng, et al. (1999). Secreted Site-1 Protease Cleaves Peptides Corresponding to Luminal Loop of Sterol Regulatory Element-binding Proteins. The Journal of Biological Chemistry 274, 22805-22812.
Cheung, H. H., Lynn Kelly, N., Liston, P., and Korneluk, R. G. (2006). Involvement of caspase-2 and caspase-9 in endoplasmic reticulum stress-induced apoptosis: a role for the IAPB. Exp Cell Res 312, 2347-2357.
Cohen, et al. (2011). Human fatty liver disease: old questions and new insights. Science 332, 1519-1523.
Colgan, et al. (2011). Endoplasmic reticulum stress and lipid dysregulation. Expert Reviews in Molecular Medicine 13.
Croze, et al. (1984). Isolation of plasma membrane, golgi apparatus, and endoplasmic reticulum fractions from single homogenates of mouse liver. J Cell Physiol 119, 46-57.
da Palma, et al. (2014). Zymogen activation and subcellular activity of subtilisin kexin isozyme 1/site 1 protease. J Biol Chem 289, 35743-35756.
DeBose-Boyd, et al. (1999). Transport-dependent proteolysis of SREBP: relocation of site-1 protease from Golgi to ER obviates the need for SREBP transport to Golgi. Cell 99, 703-712.
Espenshade, et al. (1999). Autocatalytic processing of site-1 protease removes propeptide and permits cleavage of sterol regulatory element-binding proteins. J Biol Chem 274, 22795-22804.
Farrell, et al. (2012). Liver cholesterol: is it playing possum in NASH? Am J Physiol Gastrointest Liver Physiol 303, G9-11.
Farrell, et al. (2012). NASH is an Inflammatory Disorder: Pathogenic, Prognostic and Therapeutic Implications. Gut Liver 6, 149-171.
Fava, et al. (2012). Caspase-2 ata glance. J Cell Sci 2012 125: 5911-5915.
Feldmann, et al. (2009). UCP1 ablation induces obesity and abolishes diet-induced thermogenesis in mice exempt from thermal stress by living at thermoneutrality. Cell Metab 9, 203-209.
Horton, J. D., Goldstein, J. L., and Brown, M. S. (2002). SREBPs: activators of the complete program of cholesterol and fatty acid synthesis in the liver. J Clin Invest 109, 1125-1131.
Ibrahim, S. H., Hirsova, P., Malhi, H., and Gores, G. J. (2016). Animal Models of Nonalcoholic Steatohepatitis: Eat, Delete, and Inflame. Dig Dis Sci 61, 1325-1336.
Inamine, et al. (2016). Genetic Loss of Immunoglobulin A Does Not Influence Development of Alcoholic Steatohepatitis in Mice. Alcohol Clin Exp Res.
Krumschnabel, et al. (2009). The enigma of caspase-2: the laymen's view. Cell Death and Differentiation 16, 195-207.
Kumar, S. (2009). Caspase 2 in apoptosis, the DNA damage response and timour suppression: enigma no more? Nature Review Cancer 9, 897-903.
Lambert, et al. (2014). Increased de novo lipogenesis is a distinct characteristic of individuals with nonalcoholic fatty liver disease. Gastroenterology 146, 726-735.
Larter, et al. (2008). Animal models of NASH: getting both pathology and metabolic context right. J Gastroenterol Hepatol 23, 1635-1648.
Le, et al. (2012). Effect of colesevelam on liver fat quantified by magnetic resonance in nonalcoholic steatohepatitis: a randomized controlled trial. Hepatology 56, 922-932.
Lee, et al. (2008). Regulation of hepatic lipogenesis by the transcription factor XBP1. Science 320, 1492-1496.
Lee, et al. (2004). Proteolytic activation of sterol regulatory element-binding protein induced by cellular stress through depletion of Insig-1. J Biol Chem 279, 45257-45265.
Lodhi, et al. (2012). Inhibiting adipose tissue lipogenesis reprograms thermogenesis and PPARgamma activation to decrease diet-induced obesity. Cell Metab 16, 189-201.
Logette, et al. (2005). Caspase-2, a novel lipid sensor under the control of sterol regulatory element binding protein 2. Mol Cell Biol 25, 9621-9631.
Loomba, et al. (2017). Gut Microbiome-Based Metagenomic Signature for Non-invasive Detection of Advanced Fibrosis in Human Nonalcoholic Fatty Liver Disease. Cell Metab 25, 1054-1062 e1055.
Loomba, et al. (2015). Ezetimibe for the treatment of nonalcoholic steatohepatitis: assessment by novel magnetic resonance imaging and magnetic resonance elastography in a randomized trial (MOZART trial). Hepatology 61, 1239-1250.
Lu, et al. (2014). Opposing unfolded-protein-response signals converge on death receptor 5 to control apoptosis. Science 345, 98-101.
Lumeng, et al. (2007). Obesity induces a phenotypic switch in adipose tissue macrophage polarization. J Clin Invest 117, 175-184.
Machado, et al. (2014). Reduced Lipoapoptosis, hedgehog pathway activation and fibrosis in caspase-2 deficient mice with non-alcoholic steatohepatitis. Gut 10, 1-10.
Mancini, et al. (2000). Caspase-2 is localized at the Golgi complex and cleaves golgin-160 during apoptosis. J Cell Biol 149, 603-612.
Mari, et al. (2006). Mitochondrial free cholesterol loading sensitizes to TNF- and Fas-mediated steatohepatitis. Cell Metab 4, 185-198.
Nakagawa, et al. (2014). ER stress cooperates with hypernutrition to trigger TNF dependent spontaneous HCC development. Cancer Cell 26, 331-343.
O'Reilly, et al. (2002). Caspase-2 is not required for thymocyte or neuronal apoptosis even though cleavage of caspase-2 is dependent on both Apaf-1 and caspase-9. Cell Death Differ 9, 832-841.
Osborne, T. F., and Espenshade, P. J. (2009). Evolutionary conservation and adaptation in the mechanism that regulates SREBP action: what a long, strange tRIP it's been. Genes Dev 23, 2578-2591.
Osman, et al. (2013). A novel automated image analysis method for accurate adipocyte quantification. Adipocyte 2, 160-164.
Owen, et al. (2012). Insulin stimulation of SREBP-1c processing in transgenic rat hepatocytes requires p70 S6-kinase. Proc Natl Acad Sci USA 109, 16184-16189.
Oyadomari, et al. (2008). Dephosphorylation of translation initiation factor 2alpha enhances glucose tolerance and attenuates hepatosteatosis in mice. Cell Metab 7, 520-532.
Paglialunga, S., and Dehn, C. A. (2016). Clinical assessment of hepatic de novo lipogenesis in nonalcoholic fatty liver disease. Lipids Health Dis 15, 159.
Park, et al. (2017). Magnetic Resonance Elastography vs Transient Elastography in Detection of Fibrosis and Noninvasive Measurement of Steatosis in Patients With Biopsy-Proven Nonalcoholic Fatty Liver Disease. Gastroenterology 152, 598-607 e592.
Pastorino, et al. (2008). Tumor necrosis factor-alpha can provoke cleavage and activation of sterol regulatory element-binding protein in ethanol-exposed cells via a caspase-dependent pathway that is cholesterol insensitive. J Biol Chem 283, 25638-25649.
Pekow, et al. (2007). Hepatic steatosis is associated with increased frequency of hepatocellular carcinoma in patients with hepatitis C-related cirrhosis. Cancer 109, 2490-2496.
Puri, et al. (2009). The plasma lipidomic signature of nonalcoholic steatohepatitis. Hepatology 50, 1827-1838.
Rosen, E. D., Walkey, C. J., Puigserver, P., and Speigelman, B. M. (2000). Transcriptional regulation of adipogenesis. Genes & Development 14, 1293-1307.
Sakai, et al. (1998). Molecular Identification of the Sterol-Regulated Luminal Protease that Cleaves SREBPs and Controls Lipid Composition of Animal Cells. Molecular Cell 2, 505-514.
Sanders, et al. (2016). De novo lipogenesis in the liver in health and disease: more than just a shunting yard for glucose. Biol Rev Camb Philos Soc 91, 452-468.
Sandgren, et al. (1991). Complete hepatic regeneration after somatic deletion of an albumin-plasminogen activator transgene. Cell 66, 245-256.
Sandow, et al. (2014). ER stress does not cause upregulation and activation of caspase-2 to initiate apoptosis. Cell Death Differ 21, 475-480.
Sanjana, et al. (2014). Improved vectors and genome-wide libraries for CRISPR screening. Nat Methods 11, 783-784.
Singal, et al. (2015). Hepatocellular Carcinoma From Epidemiology to Prevention: Translating Knowledge into Practice. Clin Gastroenterol Hepatol 13, 2140-2151.
Talanian, et al. (1997). Substrate Specificities of Caspase Family Proteases. Journal of Biological Chemistry 272, 9677-9682.
Tilg, et al. (2010). Evolution of inflammation in nonalcoholic fatty liver disease: the multiple parallel hits hypothesis. Hepatology 52, 1836-1846.
Upton, et al. (2012). IRE1alpha cleaves select microRNAs during ER stress to derepress translation of proapoptotic Caspase-2. Science 338, 818-822.
Wang, et al. (1996). Cleavage of sterol regulatory element binding proteins (SREBPs) by CPP32 during apoptosis. The EMBO Journal 15, 1012-1020.
Waris, et al. (2007). Hepatitis C virus induces proteolytic cleavage of sterol regulatory element binding proteins and stimulates their phosphorylation via oxidative stress. J Virol 81, 8122-8130.
Weglarz, et al. (2000). Hepatocyte transplantation into diseased mouse liver. Kinetics of parenchymal repopulation and identification of the proliferative capacity of tetraploid and octaploid hepatocytes. Am J Pathol 157, 1963-1974.
Xu, et al. (2003). Chronic inflammation in fat plays a crucial role in the development of obesity-related insulin resistance. J Clin Invest 112, 1821-1830.
Yahagi, et al. (2002). Absence of sterol regulatory element binding protein-1 (SREBP-1) ameliorates fatty livers but not obesity or insulin resistance in Lep(ob)/Lep(ob) mice. J Biol Chem 277, 19353-19357.
Yang, et al. (2001). Decreased lipid synthesis in livers of mice with disrupted Site-1 protease gene. Proc Natl Acad Sci USA 98, 13607-13612.
Zeng, et al. (2004). ATF6 modulates SREBP2-mediated lipogenesis. EMBO J 23, 950-958.
Zhang, et al. (2011). The unfolded protein response transducer IRE1alpha prevents ER stress-induced hepatic steatosis. EMBO J 30, 1357-1375.
Zhang, et al. (2014). Role of endoplasmic reticulum stress in the pathogenesis of nonalcoholic fatty liver disease. World J Gastroenterol 20, 1768-1776.

Although the invention has been described with reference to the above examples, it will be understood that modifications and variations are encompassed within the spirit and scope of the invention. Accordingly, the invention is limited only by the following claims.

1. A method of inhibiting SREBP1-mediated de novo lipogenesis (DNL) in a mammal comprising administering to the mammal an effective amount of an inhibitor of Caspase activity or expression.

2. The method of claim 1, wherein the inhibitor of Caspase activity or expression is a small molecule, peptide antisense oligonucleotide, antibody or antibody fragment.

3. (canceled)

4. The method of claim 2, wherein the inhibitor of Caspase activity or expression is an inhibitory nucleic acid that inhibits expression of casp2 or inhibits casp2 synthesis.

5. The method of claim 4, wherein the inhibitor nucleic acid is selected from the group consisting of siRNA, shRNA, gRNA, oligonucleotides, antisense RNA, and ribozymes that inhibit casp2 synthesis.

6. The method of claim 5, wherein the inhibitory nucleic acid is administered via a viral vector.

7-22. (canceled)

23. The method of claim 6, wherein the inhibitory nucleic acid encodes a gene editing system, and wherein the step of administering comprises obtaining a sample of cells from the mammal, transfecting the gene editing system into the sample of cells to inhibit casp2 synthesis, and thereafter, transplanting the transfected cells into the mammal, thereby inhibiting SREBP1-mediated DNL in the mammal.

24. The method of claim 23, wherein the gene editing system is a CRISPR system.

25. A method of inhibiting SREBP1/2 activation in a mammal fed a high fat diet comprising administering to the mammal an effective amount of an inhibitor of Caspase activity or expression.

26. The method of claim 25, wherein the inhibitor of Caspase activity or expression is a small molecule, peptide, antisense oligonucleotide, antibody or antibody fragment.

27. The method of claim 25, wherein the inhibitor of Caspase activity or expression is an inhibitory nucleic acid that inhibits expression of casp2 or inhibits casp2 synthesis.

28. The method of claim 27, wherein the inhibitor nucleic acid is selected from the group consisting of siRNA, shRNA, gRNA, oligonucleotides, antisense RNA, and ribozymes that inhibit casp2 synthesis.

29. The method of claim 28, wherein the inhibitory nucleic acid is administered via a viral vector.

30. A method of inhibiting SREBP1/2 activation in a mammalian cell stimulated with an ER stress inducer comprising contacting the stimulated mammalian cell with an inhibitor of Caspase activity or expression.

31. The method of claim 30, wherein the inhibitor of Caspase activity or expression is a small molecule, peptide, antisense oligonucleotide, antibody or antibody fragment.

32. The method of claim 31, wherein the inhibitor of Caspase activity or expression is an inhibitory nucleic acid that inhibits expression of casp2 or inhibits casp2 synthesis.

33. The method of claim 32, wherein the inhibitor nucleic acid is selected from the group consisting of siRNA, shRNA, gRNA, oligonucleotides, antisense RNA, and ribozymes that inhibit casp2 synthesis.

34. A method of inhibiting caspase-activated S1P activity or expression in a mammal in need thereof comprising administering to the mammal an effective amount of an inhibitor of Caspase activity or expression.

35. The method of claim 34, wherein the inhibitor of Caspase activity or expression is a small molecule, peptide, antisense oligonucleotide, antibody or antibody fragment.

36. The method of claim 35, wherein the inhibitor of Caspase activity or expression is an inhibitory nucleic acid selected from the group consisting of siRNA, shRNA, gRNA, oligonucleotides, antisense RNA, and ribozymes that inhibit casp2 synthesis.

37. The method of claim 36, wherein the inhibitory nucleic acid is administered via a viral vector.

38. The method of claim 34, wherein the mammal has one or more metabolic disorders selected from the group consisting of nonalcoholic steatohepatitis (NASH), nonalcoholic fatty liver disease (NAFLD), obesity, insulin resistance, and metabolic syndrome.


SEQUENCE LISTING (57 sequences)

SEQ ID NO: 1 - protein, 5 aa, Artificial Sequence
  Synthetic Construct
VDVAD

SEQ ID NO: 2 - protein, 4 aa, Artificial Sequence
  Synthetic Construct
RSLK

SEQ ID NO: 3 - protein, 4 aa, Artificial Sequence
  Synthetic Construct
RRLL

SEQ ID NO: 4 - protein, 5 aa, Artificial Sequence
  Synthetic Construct
VAVFD

SEQ ID NO: 5 - protein, 5 aa, Artificial Sequence
  Synthetic Construct
VIFSD

SEQ ID NO: 6 - protein, 5 aa, Artificial Sequence
  Synthetic Construct
VKFYD

SEQ ID NO: 7 - protein, 5 aa, Artificial Sequence
  Synthetic Construct
VLYGD

SEQ ID NO: 8 - DNA, 25 bp, Artificial Sequence
  Synthetic Construct
caccggaacgaaagacttttcgttg

SEQ ID NO: 9 - DNA, 25 bp, Artificial Sequence
  Synthetic Construct
aaaccaacgaaaagtctttcgttcc

SEQ ID NO: 10 - DNA, 25 bp, Artificial Sequence
  Synthetic Construct
caccgccgccgtccccaactgtaaa

SEQ ID NO: 11 - DNA, 25 bp, Artificial Sequence
  Synthetic Construct
aaactttacagttggggacggcggc

SEQ ID NO: 12 - DNA, 25 bp, Artificial Sequence
  Synthetic Construct
caccggctgcgtgagaagatatctc

SEQ ID NO: 13 - DNA, 25 bp, Artificial Sequence
  Synthetic Construct
aaacgagatatcttctcacgcagcc

SEQ ID NO: 14 - DNA, 20 bp, Artificial Sequence
  Synthetic Construct
ctacatgaccagaccgcaca

SEQ ID NO: 15 - DNA, 19 bp, Artificial Sequence
  Synthetic Construct
gtgccactacgcaggagtg

SEQ ID NO: 16 - DNA, 20 bp, Artificial Sequence
  Synthetic Construct
agccaaaatcagagctggaa

SEQ ID NO: 17 - DNA, 20 bp, Artificial Sequence
  Synthetic Construct
tggatcagtctggaaaagca

SEQ ID NO: 18 - DNA, 20 bp, Artificial Sequence
  Synthetic Construct
gcagaaggggagacacattt

SEQ ID NO: 19 - DNA, 21 bp, Artificial Sequence
  Synthetic Construct
ttgacatttttggtcttgtgg

SEQ ID NO: 20 - DNA, 20 bp, Artificial Sequence
  Synthetic Construct
cacagtggtgcctaccaaga

SEQ ID NO: 21 - DNA, 21 bp, Artificial Sequence
  Synthetic Construct
tgtcttttgtcaggggtcttt

SEQ ID NO: 22 - DNA, 20 bp, Artificial Sequence
  Synthetic Construct
tgtcgcagagtgtcttccaa

SEQ ID NO: 23 - DNA, 20 bp, Artificial Sequence
  Synthetic Construct
ccgtcccttctctcttcctc

SEQ ID NO: 24 - DNA, 20 bp, Artificial Sequence
  Synthetic Construct
caccctcttcaagcttttgg

SEQ ID NO: 25 - DNA, 20 bp, Artificial Sequence
  Synthetic Construct
cgaaaaacctcttggagctg

SEQ ID NO: 26 - DNA, 20 bp, Artificial Sequence
  Synthetic Construct
cttcccactaccttggctgg

SEQ ID NO: 27 - DNA, 20 bp, Artificial Sequence
  Synthetic Construct
ccagccaaggtagtgggaag

SEQ ID NO: 28 - DNA, 20 bp, Artificial Sequence
  Synthetic Construct
acggcatggatctcaaagac

SEQ ID NO: 29 - DNA, 20 bp, Artificial Sequence
  Synthetic Construct
agatagcaaatcggctgacg

SEQ ID NO: 30 - DNA, 22 bp, Artificial Sequence
  Synthetic Construct
gcactggcaagttctactgcaa

SEQ ID NO: 31 - DNA, 23 bp, Artificial Sequence
  Synthetic Construct
gtaggtgaagagaacggccttgt

SEQ ID NO: 32 - DNA, 21 bp, Artificial Sequence
  Synthetic Construct
acgacttcctctccgacctct

SEQ ID NO: 33 - DNA, 21 bp, Artificial Sequence
  Synthetic Construct
cgaggctcacgtaaccgtagt

SEQ ID NO: 34 - DNA, 22 bp, Artificial Sequence
  Synthetic Construct
tgaaagaagcggtgaaccactg

SEQ ID NO: 35 - DNA, 22 bp, Artificial Sequence
  Synthetic Construct
tggcatctctgtgtcaaccatg

SEQ ID NO: 36 - DNA, 21 bp, Artificial Sequence
  Synthetic Construct
actgccacacctccagtcatt

SEQ ID NO: 37 - DNA, 21 bp, Artificial Sequence
  Synthetic Construct
ctttgcctcactcaggattgg

SEQ ID NO: 38 - DNA, 20 bp, Artificial Sequence
  Syntehtic Construct
ggagagccctggataccaac

SEQ ID NO: 39 - DNA, 20 bp, Artificial Sequence
  Syntehtic Construct
aagttggcatggtagccctt

SEQ ID NO: 40 - DNA, 20 bp, Artificial Sequence
  Synthetic Construct
gttcagtggtgcctctgtca

SEQ ID NO: 41 - DNA, 20 bp, Artificial Sequence
  Synthetic Construct
actgggacgacatggaaaag

SEQ ID NO: 42 - DNA, 20 bp, Artificial Sequence
  Synthetic Construct
caccctcttcaagcttttgg

SEQ ID NO: 43 - DNA, 20 bp, Artificial Sequence
  Synthetic Construct
cgaaaaacctcttggagctg

SEQ ID NO: 44 - DNA, 21 bp, Artificial Sequence
  Synthetic Construct
tggagagcaccaagacagaca

SEQ ID NO: 45 - DNA, 19 bp, Artificial Sequence
  Synthetic Construct
tgccggagtcgacaatgat

SEQ ID NO: 46 - DNA, 42 bp, Artificial Sequence
  Synthetic Construct
acacgctagcaccatggcaatggcggcgccgagcgggaggtc

SEQ ID NO: 47 - DNA, 56 bp, Artificial Sequence
  Synthetic Construct
acacggatcctcaagcgtaatctggaacatcgtatgggtacgtgggtgggtagcct

SEQ ID NO: 48 - DNA, 33 bp, Artificial Sequence
  Synthetic Construct
gctgctgtttttgaaactgggctcagtgagaag

SEQ ID NO: 49 - DNA, 33 bp, Artificial Sequence
  Synthetic Construct
ttcaaaaacagcagctctgacattagcacctgt

SEQ ID NO: 50 - DNA, 33 bp, Artificial Sequence
  Synthetic Construct
gccatcttcagtgagtggtacaacacttctgtt

SEQ ID NO: 51 - DNA, 33 bp, Artificial Sequence
  Synthetic Construct
ctcactgaagatggcgagggaaaggccattgtc

SEQ ID NO: 52 - DNA, 33 bp, Artificial Sequence
  Synthetic Construct
gcgaagttttatgaggaaaacaccaggcagtgg

SEQ ID NO: 53 - DNA, 33 bp, Artificial Sequence
  Synthetic Construct
ctcataaaacttcgcttttctcataacagaagt

SEQ ID NO: 54 - DNA, 33 bp, Artificial Sequence
  Synthetic Construct
gcgctgtatggagagtccaactgcttggatgac

SEQ ID NO: 55 - DNA, 33 bp, Artificial Sequence
  Synthetic Construct
ctctccatacagcgcgatccggcctccaccttc

SEQ ID NO: 56 - DNA, 33 bp, Artificial Sequence
  Synthetic Construct
caagcaggtcgtggagatgagacagatagaggt

SEQ ID NO: 57 - DNA, 33 bp, Artificial Sequence
  Synthetic Construct
tccacgacctgcttggatgaagaacatttttgg